(19) United States
(12) Patent Application Publication
(10) Pub. No.: US 2008/0153152 A1
(43) Pub. Date: Jun. 26, 2008

(54) MICROFLUIDIC CHIP

(76) Inventors: Akira Wakabayashi, Minami-Ashigara-shi (JP); Hideyuki Karaki, Minami-Ashigara-shi (JP); Yoshihiro Sawayashiki, Minami-Ashigara-shi (JP); Kota Kato, Minami-Ashigara-shi (JP); Yoshihide Iwaki, Ashigarakami-gun (JP)

Correspondence Address: BIRCH STEWART KOLASCH & BIRCH, PO BOX 747, FALLS CHURCH, VA, 22040-0747, US

(21) Appl. No.: 11/943,380

(22) Filed: Nov. 20, 2007

(30) Foreign Application Priority Data
Nov. 22, 2006 (JP) P2006-316129
Nov. 15, 2007 (JP) P2007-297003

(51) Int. Cl. C12M 1/00

(52) U.S. Cl. 435/287.2

(57) ABSTRACT

A microfluidic chip, includes: a first port for inputting: a sample liquid; and a first liquid; a second port for inputting a second liquid; a third port for supplying air pressure; a first channel (A) for mixing the sample liquid and the first liquid to generate a first mixed liquid; a second channel (B) for beating the first mixed liquid; a third channel (C) for allowing the second liquid to converge into the first mixed liquid to generate a second mixed liquid; a fourth channel (D) installing a first solid; a fifth channel (E) for promoting mixing of the first solid; a plurality of sixth channels (F) each having a second solid; and a seventh channel (G), which connects the fifth channel (E) and the plurality of sixth channels (F), for dispensing a fixed quantity of the second mixed liquid to each of the plurality of sixth channels (F).

### BACKGROUND OF THE INVENTION

1. Field of the Invention

This invention relates to a microfluidic chip for analyzing biological materials, such as blood, etc.

2. Description of the Related Art

The recent progress of molecular biology has indicated that the effect of medicine administration in disease treatment and an individual difference of a side effect caused by a constitutional predisposition can be predicted by analyzing biological materials, such as blood, etc., and using this, there has been a growing trend to conduct optimum treatment for each individual. For example, if it is known that there is strong correlation between a specific gene and the effect and side effect of a specific curative medicine, to make the information useful for treatment of a specific patient, the base sequence of the genes of the patient needs to be known. Gene diagnosis to obtain information concerning mutation in endogenous genes or single nucleotide polymorphism (SNP) can be conducted by amplification and detection of a target nucleic acid containing such mutation or single nucleotide polymorphism. Thus, a simple and easy method of capable of amplifying and detecting the target nucleic acid in a sample rapidly and precisely is demanded.

In this case, protein of an antibody, an antigen, or the like bound specifically with a detected substance or a single-stranded nucleic acid is used as a probe and is fixed to a solidus surface of fine particles, beads, glass plate, etc., and antigen-antibody reaction or nucleic acid hybridization is executed with the detected substance. Using a labeled substance having a specific interaction carrying a label substance having high detection sensitivity such as an enzyme, for example, a labeled antibody, a labeled antigen, a labeled nucleic acid, or the like, an antigen-antibody compound or a double stranded nucleic acid is detected and the presence or absence of the detected substance is detected or the detected substance is quantified.

As this kind of art, for example, a bacterial spore treatment chip and a bacteria spore treatment device disclosed in JP-A-2005-253365 include a pour hole to which a specimen containing bacteria forming spores is supplied, a germination promotion liquid storage section for storing a germination promoter introduced into the specimen supplied to the pour hole, a lysis solution storage section for storing a lysis solution introduced into mixed liquid of the specimen and the germination promoter, a gene elution section for mixing the specimen, the germination promoter, and the lysis solution and eluting genes from the specimen, a gene extraction section including a gene bind carrier bound with the eluted genes, a cleaning liquid storage section for storing cleaning liquid introduced into the gene extraction section, an eluting solution storage section for storing an eluting solution introduced into the gene extraction section, and a reaction section into which the genes eluted by means of the eluting solution are introduced; they are easy to handle and are inexpensive and make it possible to automate the steps of bacteria spores to extraction and analysis of genes collectively.

A nucleic acid amplification substrate disclosed in JPA-2006-115741 has a first layer made of a glass plate and a second layer made of silicon rubber, which are deposited on each other The second layer is formed on the contact face with the first layer with minute grooves to form a gap between the first layer and the second layer. In the nucleic acid amplification substrate, four channels of nucleic acid amplification and separation channels are formed according to the gap pattern (groove pattern) The nucleic acid amplification and separation channel has a reaction liquid reservoir section and an electrophoresis section and on the periphery thereof, a reaction liquid going and returning channel, a reaction liquid suction channel, and a nucleic acid supply channel are provided. The reaction liquid reservoir section is provided with a zigzag and labyrinthine channel in a gap shaped roughly like a square on a front view. Accordingly, reaction liquid is prevented from leaking in a nucleic acid amplification reaction step in the nucleic acid amplification substrate for conducting nucleic acid amplification reaction of PCR, etc, on the substrate.

Further, as a biological material testing device disclosed in JP-A-2006-125990, a chip component of microreactor for each sample installing reagent and liquid delivery elements and a control and detection component of the device main body are configured as separate systems, whereby cross contamination and carry-over contamination are made hard to occur for ultramicroanalysis and amplification reaction.

### SUMMARY OF THE INVENTION

A DNA sequencer, an DNA microarray, and the like are known as methods in the related arts to know the base sequence of specific genes; however, they involve various problems of generally high costs of devices and chips, long time required for testing, etc. That is, in JP-A-2005-253365, extraction and analysis of genes can be automated collectively, but only a single item can be detected; this is a problem.

In JP-A-2006-115741, although a plurality of items can be tested collectively, extraction of genes, dispensing of a sample, and preparations for a reagent required for amplification need to be performed as pretreatment and to perform manual operation, the operation is intricate and skill is also required; it is teared that erroneous results may be obtained due to contamination, etc., caused by erroneous operation. To perform automatic operation, the device becomes complicated and upsized and becomes expensive; this is a problem.

Further, JP-A-2006-125990 discloses the chip capable of testing a plurality of items collectively; however, a complicated stereoscopic structure of a liquid delivery control section for permitting passage of liquid if a preset pressure is reached, a back flow check section for preventing a back flow of liquid in a channel, and the like is required and the circuit is very complicated and if an attempt is made to test multiple items, the chip becomes expensive; this is a problem.

On the other hand, JP-A-2005-160387 proposes a nucleic acid amplification method and a nucleic acid amplification primer set for amplifying only a target gene and analyzing it in a comparatively easy and simple detection system. To perform the reaction according to a method of pipette operation using a microtube used with general biochemical analysis, operation of preparations for reagents, operation of pipetting, taking out from taking to the device, etc., is complicated and skill is also required; this is a problem. Particularly, to analyze a plurality of target genes, the risks of taking an erroneous liquid medicine and contamination because of intricate operation increase and reliability of the testing result is insufficient, this is a problem.

It is therefore an object of the invention to provide a microfluidic chip capable of providing the precise and highly reliable analysis result at a low cost and in a short time by performing simple operation requiring no skill.

The object of the invention is accomplished by the following configurations:

(1) A microfluidic chip including channels for detecting a plurality of types of nucleic acid sequences, comprising:

a first port for inputting: a sample liquid containing a biological cell; and a first liquid;

a second port for inputting a second liquid:

a third port for supplying air pressure to the channels;

a first channel (A) for mixing the sample liquid and the first liquid input from the first port to generate a first mixed liquid;

a second channel (B) for heating the first mixed liquid;

a third channel (C) for allowing the second liquid to converge into the first mixed liquid treated in the second channel (B) to generate a second mixed liquid;

a fourth channel (D) installing a first solid that dissolves with the passage of the second mixed liquid converged in the third channel (C);

a fifth channel (E) for promoting mixing of the first solid into the second mixed liquid treated in the fourth channel (D);

a plurality of sixth channels (F) each having a second solid solidified and installed in the sixth channel (F); and

a seventh channel (C), which connects the fifth channel (E) and the plurality of sixth channels (F), for dispensing a fixed quantity of the second mixed liquid treated in the fifth channel (E) to each of the plurality of sixth channels (F).

The microfluidic chip includes channels for mixing with various reagents and dispensing a fixed quantity of the mixed liquid as component measures in addition to the first port for inputting sample liquid and first liquid, the second port for inputting second liquid, and the third port for supplying air pressure to the channel, whereby it is made possible to perform complicated handling of limited liquid by pneumatic drive from the outside of the chip particularly with simple channels not containing any active valve or pump. This means that liquid delivery control is made possible according to a simple structure without requiring a stereoscopically complicated structure Accordingly, simply by inputting a sample and a liquid reagent, automatically any desired droplet operation and chemical reaction are conducted and the need for intricate operation of pipetting, taking out from, taking to the device, etc., is eliminated and the high analysis result can be obtained.

(2) The microfluidic chip as described in (1) above,

wherein the first liquid comprises a pretreatment reagent.

According to the microfluidic chip, the sample liquid and the pretreatment reagent are mixed.

(3) The microfluidic chip as described in (1) or (2) above,

wherein the second liquid comprises a reaction amplification reagent.

According to the microfluidic chip, the reaction amplification reagent is mixed in the first mixed liquid

(4) The microfluidic chip as described in any of (1) to (3) above,

wherein an enzyme is mixed into the first solid.

According to the microfluidic chip, the enzyme dissolves with the passage of the second liquid,

(5) The microfluidic chip as described in any of (1) to (4) above,

wherein a primer is mixed into the second solid.

According to the microfluidic chip, the primer is mixed in the second solid, whereby DNA amplification is executed.

(6) The microfluidic chip as described in any of (1) to (5) above, which is a microfluidic chip for detecting presence or absence of a plurality of types of nucleic acid sequences contained in a blood.

According to the microfluidic chip, blood (whole blood) is used as a sample and the target nucleic acid is amplified and is detected, whereby it is made possible to amplify and detect the target nucleic acid specific to the pathogen causing an infectious disease, and it is made possible to determine whether or not the pathogen exists in the sample.

(7) The microfluidic chip as described in any of (1) to (6) above, which is a microfluidic chip for detecting presence or absence of a single nucleotide polymorphism.

According to the microfluidic chip, blood (whole blood) is used as a sample and reaction to amplify the nucleic acid of the target sequence specifically and detection thereof are executed on the microfluidic chip and it is made possible to test a plurality of target genes of single nucleotide polymorphism type.

(8) The microfluidic chip as described in any of (1) to (7) above,

wherein DNA amplification reaction is executed isothermally in the sixth channel (F).

According to the microfluidic chip, the DNA amplification reaction is kept at a temperature at which the activity of the used enzyme can be maintained constant by isothermal amplification reaction. The term “isothermal” mentioned here refers to such an almost constant temperature at which an enzyme and a primer can function substantially. Further, the expression “almost constant temperature” is used to mean that temperature change to such an extent that the substantial function of an enzyme and a primer is not impaired is allowed.

(9) The microfluidic chip as described in any of (1) to (8) above, which has light-transmitting property enable to detect fluorescence occurring in the DNA amplification.

Since the microfluidic chip has light-transmitting property, for example, cybergreen is used for a detection reagent and it is made possible to measure fluorescence emitted as it is intercalated into double stranded DNA amplified by reaction. Accordingly, it is made possible to detect the presence or absence of a gene sequence as a target.

(10) The microfluidic as described in any of (1) to (9) above,

wherein the first channel (A) comprises an alternating pattern of: wide channel parts each with a cross-section area in an orthogonal direction to a flow direction of a liquid being larger than cross-sectional areas in any other channels in the first channel (A); and narrow channel parts each having a smaller cross-sectional area than the wide channel parts.

According to the microfluidic chip, when the blood input to the first port reaches the first channel, the blood and the pretreatment reagent pass through the channel formed with the alternating pattern of the wide channel parts and the narrow channel parts, whereby agitation of orifice effect is performed more than once and the blood and the pretreatment reagent are mixed uniformly.

(11) The microfluidic chip as described in any of (1) to (10) above,

wherein the third channel (C) comprises:

a port for retaining the second liquid;

a main channel where the first mixed liquid is delivered; and

a port exit channel disposed at a midpoint in the main channel for allowing the main channel to communicate with the port, and

wherein magnitude relation of capillary forces is: port exit channel>main channel>port.

According to the microfluidic chip, the connection part of the port exit channel and the main channel forms a Laplace pressure valve and the reaction amplification reagent converges with the blood and the pretreatment reagent subjected to heating treatment. That is, the reaction amplification reagent input to the port remains on the connection face of the port exit channel and the main channel without flowing out to the main channel. When the mixed liquid of the blood and the pretreatment reagent arrives at the port exit channel, the Laplace pressure valve is destroyed and the two liquids converge.

(12) The microfluidic chip as described in any of (1) to (11) above,

wherein the fourth channel (D) comprises:

a retention section for installing the first solid; and

channels placed in upstream and downstream sides of the retention section each having a narrower width than the retention section.

According to the microfluidic chip, the channels upstream and downstream from the retention section are thinner than the retention section, thereby preventing the solidified reagent from peeling off and flowing out to the preceding or following channel due to vibration of retention, transport, etc., of the chip if there is no adhesion of the dried and solidified reagent to the channel.

(13) The microfluidic chip as described in any of (1) to (12) above,

wherein the fifth channel (E) comprises a plurality of liquid reservoir chambers, and

the fourth channel (D) is disposed between the plurality of liquid reservoir chambers, and

wherein the second mixed liquid goes and returns between the plurality of liquid reservoir chambers, so as to dissolve and mix the first solid.

According to the microfluidic chip, the mixed liquid of the blood, the pretreatment liquid, and the reaction amplification reagent go and return between the plurality of liquid reservoir chambers, whereby enzyme 1 and enzyme 2 dissolve and the enzyme 1 and the enzyme 2 and the mixed liquid are mixed uniformly.

(14) The microfluidic chip as described in any of (1) to (13) above,

wherein the fifth channel is provided at a midpoint in a channel from the first and second ports to the third port and comprises a first mixing section and a second mixing section placed in order from a side of the first and second ports, and

wherein each of the first mixing section and the second mixing section is formed alternately with: first channel parts each with a perpendicular cross-section area in a flow direction of a liquid being larger than perpendicular cross-sectional areas in any other channels in each of the first mixing section and the second mixing section; and second channel parts each having a smaller perpendicular cross-sectional area than the first channel part,

the perpendicular cross-sectional area of the first channel part in the first mixing section is formed smaller than that of the first channel part in the second mixing section, and

a channel direction length of the first channel part in the first mixing section is formed longer than a channel direction length of the first channel part in the second mixing section.

According to the microfluidic chip, the perpendicular cross-sectional area of the first channel part in the first mixing section is formed smaller than that of the first channel part in the second mixing section and the channel direction length of the first channel part in the first mixing section is formed longer than the channel direction length of the first channel part in the second mixing section, so that a plurality of types of liquids are preliminarily mixed in the first mixing section wherein a difference is hard to occur in the proceeding degree of the meniscus curved surface liquid end. In the second mixing section high in mixing performance, mixing treatment can be executed while suppressing the difference in the proceeding degree of the meniscus curved surface liquid end caused by the tact that liquids different in wettability come in contact with the channel face. Accordingly, if liquids different in wettability are introduced into the mixing section, a liquid unfilled part (air bubble) is not formed in the mixing section and a plurality of liquids or solid lysis solutions different in liquid physical properties can be mixed stably,

(15) The microfluidic chip as described in any of (1) to (14) above,

wherein the second solid in the sixth channel (F) is solidified and placed on an upper face of the sixth channel (F).

According to the microfluidic chip, in a microchip use state, the second solid is placed on the upper face of the channel and is heated from the lower face, whereby the second solid dissolved with temperature rise of the liquid flows to the lower side in the channel by gravity when the gravity of the second solid is large.

(16) The microfluidic chip as described in any of (1) to (15) above,

wherein the primer placed in the sixth channel (F) is mixed in a substance dissolved by heating and is solidified.

According to the microfluidic chip, the primer is mixed with a substance dissolved by heating, for example, gelatin and is solidified. The primer and the gelatin are mixed and diffused uniformly in a short time in the cells because of the multiplier effect of the flow to the lower face of the channel caused by the gravity of the gelatin and the convection caused by heating the liquid.

(17) The microfluidic chip as described in any of (1) to (16) above,

wherein the sixth channel (F) comprises:

a reaction detection cell for retaining the primer; and

an upstream channel and a downstream channel of the reaction detection cell, and

wherein a heated region consisting of the whole reaction detection cell and parts of channels in a reaction detection cell side of the upstream channel and the downstream channel is formed thinner than any other regions in the sixth channel (F).

According to the microfluidic chip, the heated region is formed thinner than any other region and uniform heating is made possible.

(18) The microfluidic chip as described in any of (1) to (17) above, further comprising:

a block member for blocking all of the first, second, and third ports to perform amplification reaction using the reaction amplification reagent in a hermetically sealed space.

According to the microfluidic chip, before amplification reaction, the block member blocks all of the first, second, and third ports and the amplification reaction is performed in the hermetically sealed state of the chip. Accordingly, the following risk is avoided: If amplification reaction is performed in a state in which the chip is not hermetically sealed, the amplified DNA flows out to the outside of the chip, contaminating the environment and causing carry over to occur

(19) The microfluidic chip as described in any of (1) to (18) above,

wherein an inner face of the sixth channel (F) is a continuous smooth face for preventing formation of a minute gap space not filled with liquid when a liquid flows through an inside of the sixth channel (F).

According to the microfluidic chip, the inner face of the channel is a continuous smooth face not formed with any minute gap space, and an air bubble is prevented from occurring in the channel at the heating time. Accordingly, degradation of the fluorescence detection accuracy is prevented.

(20) The microfluidic chip as described in any of (1) to (19) above,

wherein an inner face of each of the channels has wettability of at least two levels or more.

According to the microfluidic chip, resins different in wettability are used, the inner faces of the channels of the molded channel substrate are made hydrophilic or water-repellent, liquid smoothly enters at the dispensing time to the reaction detection cells, and stop with the Laplace pressure valve at the exit can be stably performed.

The microfluidic chip according to the invention includes the first port for inputting sample liquid containing biological cells and first liquid; the second port for inputting second liquid; the third port for supplying air pressure to the channel; the first channel (A) for mixing the sample liquid and the first liquid input from the first port to generate first mixed liquid; the second channel (B) for heating the first mixed liquid; the third channel (C) for allowing the second liquid to converge into the first mixed liquid treated in the second channel (B); the fourth channel (D) installing a first solid dissolving with the passage of the second mixed liquid converged in the third channel (C); the fifth channel (E) for promoting mixing of the first solid into the second mixed liquid treated in the fourth channel (D); the sixth channel (F) connected to the fifth channel (E) and having a second solid solidified and installed in the channel; and the seventh channel (G) connected to the plurality of sixth channels for dispensing a fixed quantity of the second mixed liquid treated in the fifth channel (E) to each of the plurality of sixth channels (F), so that liquid delivery control can be performed according to the simple structure without requiring a stereoscopically complicated structure, the need for intricate operation of pipetting, taking out from, taking to the device, etc., is eliminated, and the precise and highly reliable analysis result can be provided at a low cost and in a short time by performing simple operation requiring no skill.

### BRIEF DESCRIPTION OF THE DRAWINGS

In the accompanying drawings:

FIG. 1 is a block diagram to represent the microfluidic chip according to the invention together with the schematic configuration of a testing apparatus;

FIG. 2 is an exploded perspective view of the microfluidic chip shown in FIG. 1;

FIG. 3A is a plan view of the microfluidic chip as a top view and FIG. 3B is a plan view of the microfluidic chip as a bottom view;

FIG. 4 is an enlarged view of FIG. 3B;

FIG. 5 is an exploded perspective view to represent the lower face of the chip before block members are put;

FIG. 6 is a main part enlarged plan view to represent the proximity of a port exit channel;

FIG. 7 is an enlarged view of a first mixing section and a second mixing section;

FIGS. 8A and 8B are schematic representations to show the relationships between the width of a first channel part and the meniscus length;

FIG. 9A in a sectional view taken on line P2-P2 in FIG. 4 where primers are mixed and diffused and FIG. 9B is a schematic representation of a reaction detection cell as a main part enlarged view;

FIG. 10 is an enlarged plan view of the reaction detection cells;

FIG. 11A is a graph of the fluorescence measurement result when a target sequence exists and FIG. 11B is a graph of the fluorescence measurement result when a target sequence does not exist;

FIGS. 12A to C are plan views to represent the foaming situation of a reaction detection section;

FIG. 13 is main part sectional views to represent foaming prevention measures of the reaction detection section;

FIG. 14A is a plan view of a liquid position detection section and FIG. 14B is a sectional view taken on line P1-P1 in FIG. 14A;

FIG. 15 in a schematic drawing to represent incidence light and reflected light of the liquid position detection section;

FIG. 16 is a graph to represent the correlation between reflectivity and incidence angle;

FIG. 17 is a side view of the liquid position detection section wherein a light emission optical fiber and a light reception optical fiber are placed as they are inclined;

FIG. 18 is a time chart to represent the operation state of each component involved in the drive control of the microfluidic chip along the time axis,

FIG. 19 is a schematic representation of the operation from liquid setting to the first heating (s1) to (s6);

FIG. 20 is a schematic representation of the operation from convergence of second liquid to enzyme mixing (s7) to (s12);

FIG. 21 is a schematic representation of the operation from mixing treatment to dispensing into a reaction section (s13) to (s18);

FIG. 22 is a schematic representation of the operation of completion of dispensing (s19);

FIG. 23 is a plan view to represent the bottom view of a microfluidic chip;

FIG. 24 is a time chart to represent the operation state of each component involved in the drive control of the microfluidic chip along the time axis;

FIG. 25 is a schematic representation of the operation from liquid setting to the first heating;

FIG. 26 is a schematic representation of the operation to enzyme mixing;

FIG. 27 is a schematic representation of the operation to dispensing into a reaction section; and

FIG. 28 is a schematic representation of the operation from dispensing to testing completion.

### DETAILED DESCRIPTION OF THE INVENTION

Preferred embodiments of a microfluidic chip according to the invention will be discussed in detail with reference to the accompanying drawings.

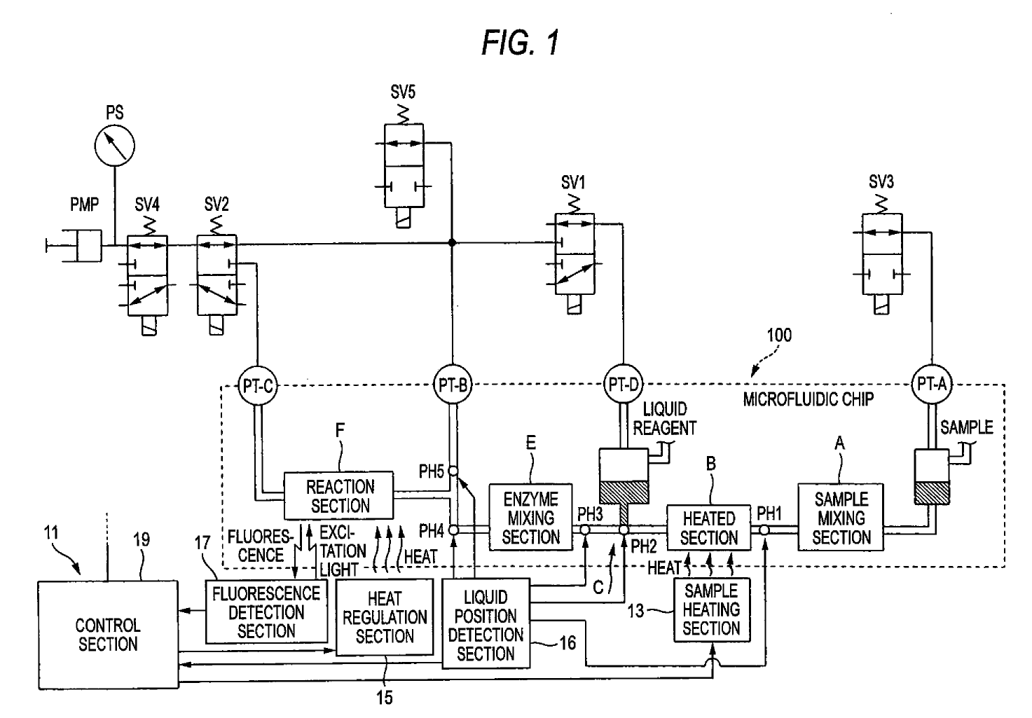

FIG. 1 is a block diagram to represent the microfluidic chip according to the invention together with the schematic configuration of a testing apparatus.

A microfluidic chip (also simply called “chip”) 100 according to a first exemplary embodiment of the invention is set in a testing apparatus 11 for use and is discarded after once used. In the embodiment, blood (whole blood) of a sample is poured into the microfluidic chip 100. The microfluidic chip 100 is set in the testing apparatus 11, whereby the sample liquid is handled by a physical action force from the outside of the chip and, for example, a plurality of target genes of single nucleotide polymorphisms are tested; reaction to amplify the nucleic acid of the target sequence isothermally and specifically and detection thereof can be realized on the chip 100 as shown in JP-A-2005-160387. Accordingly, for example, the target nucleic acid is amplified and is detected, whereby it is made possible to amplify and detect the target nucleic acid specific to the pathogen causing an infectious disease, and it is made possible to determine whether or not the pathogen exists in the sample, etc.

In the embodiment, the physical action force is a pneumatic action force (pneumatic drive force) generated by air supply or air suction from a port part PT provided at the start point and the end point of a liquid channel. Therefore, it is made possible to perform move control of liquid supplied to the liquid channel to any desired position in the liquid channel by air supply or air suction acted on the start point and the end point of the liquid channel. At this time, the liquid is held in a state in which it is clamped in the gas intervening between the start point and the tip part and between the rear end part and the end point and is not broken midway by the action of a tensile force.

The DNA amplification reaction is kept at a temperature at which the activity of the used enzyme can be maintained constant by isothermal amplification reaction. The term “isothermal” mentioned here refers to such an almost constant temperature at which an enzyme and a primer can function substantially. Further, the expression “almost constant temperature” is used to mean that temperature change to such an extent that the substantial function of an enzyme and a primer is not impaired is allowed.

The testing apparatus 11 is provided with basic components of a pump PMP using air as a working fluid, valves SV1, SV2, SV3, SV4, and SV5, a sample heating section 13, a heat regulation section 15, a liquid position detection section 16, a fluorescence detection section 17, and a control section 19 connected to the components for inputting a detection signal or sending a control signal. A pressure sensor is provided between the pump PMP and the valve SV4. The valve SV4 is intervened between the pump PMP and the valve SV2, the valve SV2 is connected on the working fluid control side to a fourth port PT-C of the chip 100, the valve SV1 is connected on the working fluid control side to a second port PT-D of the chip 100, the valve SV3 is connected on the working fluid control side to a first port PT-A of the chip 100, and the working fluid control side of the valve SV2 and the working fluid input side of the valve SV1 are connected to a third port PT-B of the chip 100. The sample heating section 13 heats a heated section B of the chip 100, the heat regulation section 15 performs temperature control of a reaction section F of the chip 100, and the fluorescence detection section 17 can detect fluorescence of the reaction section F. The operation of the components is described later in detail.

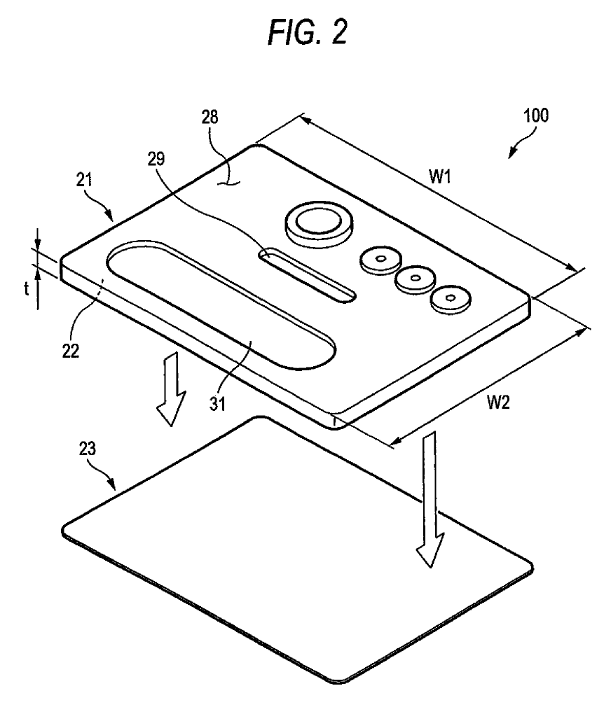

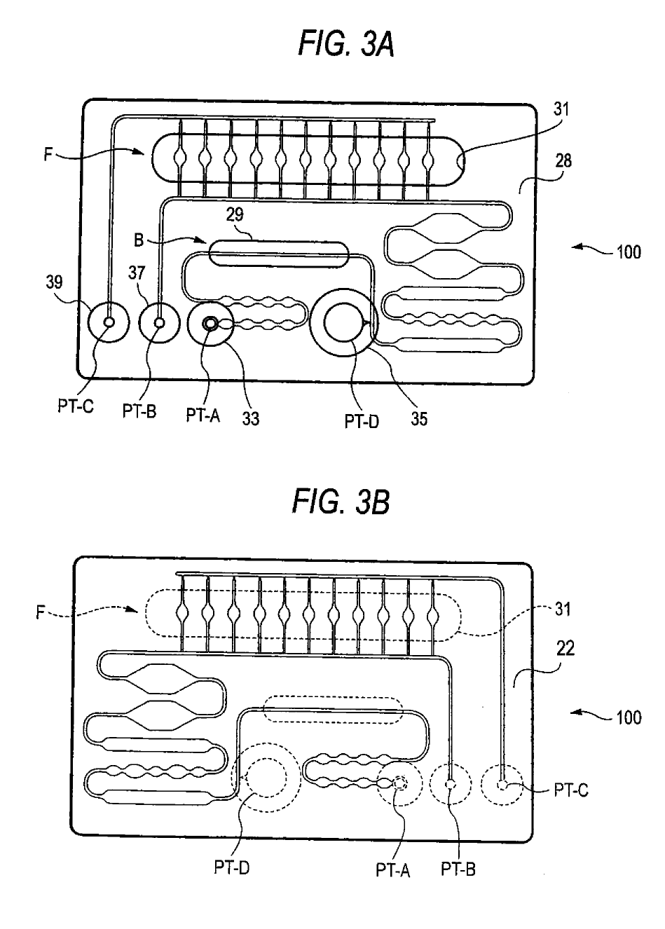

FIG. 2 is an exploded perspective view of the microfluidic chip shown in FIG. 1 and FIG. 3A is a plan view of the microfluidic chip as a top view and FIG. 3B is a plan view of the microfluidic chip as a bottom view.

The microfluidic chip 100 is made up of a channel substrate 21 and a lid 23 put on one face (lower face) 22 of the channel substrate 21, as shown in FIG. 2. The channel substrate 21 is manufactured by injection molding of a thermoplastic polymer. Although the polymer to be used is not limited, it is desirable that the polymer should be optically transparent, have high heat resistance, be chemically stable, and be easily injection molded; COP, COC, PMMA, etc., is preferred. The expression “optically transparent” is used to mean that transmittance is high in the wavelengths of excitation light and fluorescence used for detection, that scattering is small, and autofluorescence is small. Since the chip 100 has light-transmitting property for making it possible to detect fluorescence, for example, cybergreen is used for a detection reagent and it is made possible to measure fluorescence emitted as it is intercalated into double stranded DNA amplified by reaction. Accordingly, it is made possible to detect the presence or absence of a gene sequence as a target.

The channel substrate 21 is formed on an opposite face (upper face) 28 with excavations 29 and 31, which are positioned corresponding to the heated section B and the reaction section F. Openings 33, 35, 37, and 39 communicating with the first port PT-A, the second port PT-D, the third port PT-B, and the fourth port PT-C are made in the lower face 22 of the channel substrate 21 as shown in FIGS. 3A and 3B. The channel substrate 21 is formed, for example, as dimensions of 55×91 mm of length W2×width W1 and having a thickness t of about 2 mm

The lid 23 is a member for lidding the ports, the cells, and the channels (grooves) formed on the channel face (lower face 22) of the channel substrate 21, and the lid 23 and the channel substrate 21 are joined with an adhesive or a pressure sensitive adhesive. A sheet-like polymer which is optically transparent, has high heat resistance, and is chemically stable is used as the lid 23 like the channel substrate. In the embodiment, a PCR plate seal having a thickness of 100 μm is used (a pressure sensitive adhesive is applied to a plastic film).

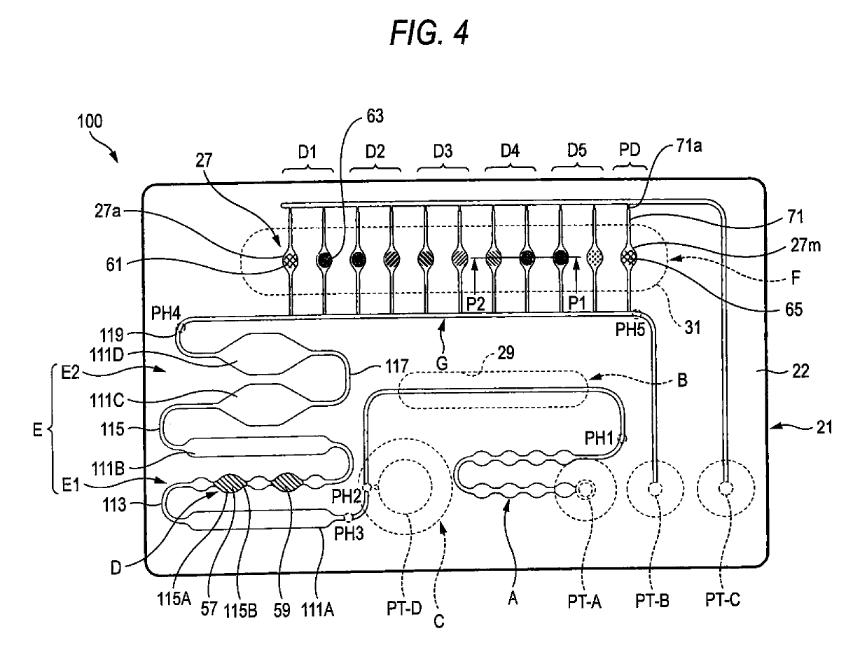

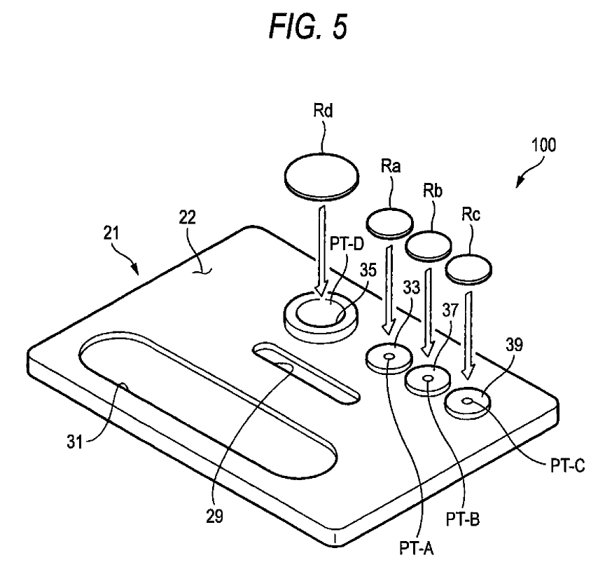

FIG. 4 is an enlarged view of FIG. 33, FIG. 5 is an exploded perspective view to represent the lower face of the chip before block members are put, and FIG. S is a main part enlarged plan view to represent the proximity of a port exit channel The channel substrate 21 is formed with the ports, the cells, the channels, etc., for performing necessary operation on liquid (described later in detail). That is, the channel substrate 21 includes the first port PT-A for inputting sample liquid containing biological cells and a pretreatment reagent (first liquid), the second port PT-D for inputting a reaction amplification reagent (second liquid), the third port PT-B for supplying air pressure to the channel, the fourth port PT-C at the channel termination where pressure is reduced, a first channel (sample mixing section) A for mixing the sample liquid and the pretreatment reagent input from the first port PT-A to generate first mixed liquid, a second channel (heated section) B for heating the first mixed liquid, extracting DNA from the biological cell, and decomposing the DNA into a single strand, a third channel (reagent converging section) C for allowing the reaction amplification reagent to converge into the first mixed liquid treated in the heated section B, a fourth channel (enzyme retention section) D solidifying and installing an enzyme (first solid) whose dissolution advances with the passage of the second mixed liquid converged in the reagent converging section C, a fifth channel (enzyme mixing section) E for promoting mixing of the enzyme into the second mixed liquid treated in the enzyme retention section D, a plurality of sixth channels (reaction section) F connected to the enzyme mixing section E for executing DNA amplification by dissolving and heating a primer (second solid) solidified and installed i n the channel and detection of DNA amplification at the same position, and a seventh channel (fixed-quantity dispensing channel) G connected to the channel of the reaction section F for dispensing a fixed quantity of the second mixed liquid treated in the enzyme mixing section E to each of a plurality of reaction detection cells 27 of the reaction section F, as shown in FIG. 4.

The first port PT-A, the second port PT-D, the third port PT-B, and the fourth port PT-C (port section PT) are made of holes piercing the top and bottom faces of the channel substrate 21 and the lid 23 is put thereon, whereby concave parts communicating with the channels are formed. Each port section PT is made a slightly thicker than any other portion of the channel substrate 21 and a liquid delivery port pad (not shown) of the testing apparatus 11 is connected thereto. Each port pad is connected via piping to the valves SV1, SV2, SV3, and SV4 (valve SV). The above-mentioned pump PMP for liquid delivery drive is connected to the valve SV. The control section 19 can control the operation of the valve SV and the pump PMP, thereby placing the air of the port section PT in a reduced pressure state, a pressurization state, an atmospheric release state, or a hermetically sealed state and transporting droplets in the channel as desired.

Upon completion of any desired transport, the port pads are detached from the port sections PT and labels Ra, Rb, Rc, and Rd shown in FIG. 5 are put, whereby the microfluidic chip 100 can be placed in a hermetically sealed state. If amplification reaction is executed in a state in which the chip 100 is not hermetically sealed, there is the risk of allowing amplified DNA to flow out from the chip, polluting the environment, and causing carry over. To prevent this, the chip 100 is placed in the hermetically sealed state before amplification reaction. As methods for hermetically sealing the chip 100, a method of putting a cap having hermaticity (not shown) on the port section PT or any other known sealing method such as a method of pouring UV cure resin into the port section PT and then irradiating it with UV light for solidification can be used in addition to the method of putting the label Ra, Rb, Rc, Rd mentioned above.

The first port PT-A is used as a sample port and blood 1 μL and pretreatment reagent 3 μL are input thereto. The pretreatment reagent is used to isolate a nucleic acid component from leucocytes in blood. Chemical dissolving treatment is performed using a surfactant or strong alkali. For example, a nonionic surfactant, a cationic surfactant, an anionic surfactant, an ampholytic surfactant, etc., can be named as the surfactant. To prevent blood coagulation, an anticoagulant of heparin, EDTA, etc., may be added.

The second port PT-D is used as a liquid reagent port and a reaction amplification reagent (56 μL) is input thereto. The reaction amplification reagent contains a reagent required for amplification reaction and detection other than an enzyme, a primer. For example, a catalyst of magnesium chloride, magnesium acetate, magnesium sulfate, etc., a substrate of dNTP mix, etc., a buffer solution of a tris hydrochloride buffer, a tricine buffer, a sodium biphosphate buffer, a potassium dihydrogen phosphate buffer, etc., can he used. Further, an additive of dimethyl sulfoxide, betaine (N,N,N-trimethylglycine), etc., an acid substance, a cationic complex, etc, described in International Patent Publication No. 99/54455 pamphlet may be used.

Cybergreen can be used as the detection reagent Cybergreen is intercalated into double stranded DNA amplified by reaction, whereby it emits strong fluorescence. The fluorescence strength is measured, whereby the presence or absence of a gene sequence as a target is detected.

The third port PT-B and the fourth port PT-C are used as liquid delivery ports and are switched to a reduced pressure state, a pressurization state, an atmospheric release state, a closed sate by the pump PMP and the valve SV, thereby driving droplets in the channel.

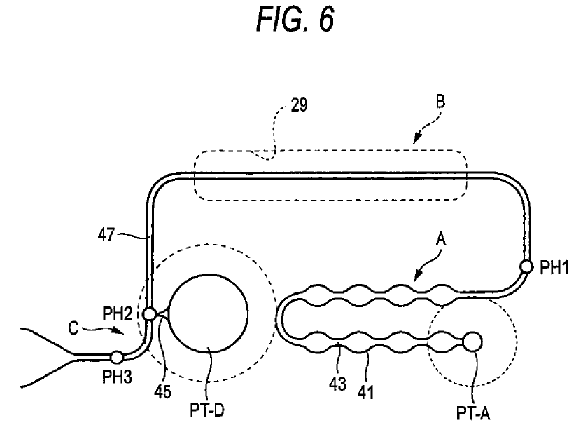

As shown in FIG. 6, the sample mixing section A is a channel with a concatenation of tortoiseshell-shaped cells larger than the whole amount of the blood and the pretreatment reagent input to the first port PT-A and the blood and the pretreatment reagent are allowed to pass through the channel, the blood and the pretreatment reagent input to the first port PT-A are mixed uniformly. That is, the channel of the sample mixing section A is formed with an alternating pattern of wide channel parts 41 each with the cross-section area in an orthogonal direction to the flow direction of liquid being larger than the cross-sectional area in any other channel and narrow channel parts 43 each having a smaller cross-sectional area than the wide channel part 41. Therefore, when the blood input to the first port PT-A reaches the sample mixing section A, the blood and the pretreatment reagent pass through the channel formed with the alternating pattern of the wide channel parts 41 and the narrow channel parts 43 along the liquid flowing direction, whereby agitation of orifice effect is performed more than once and the blood and the pretreatment reagent are mixed uniformly.

The heated section B is heated to 98° C. by the sample heating section 13 shown in FIG. 1. That is, in the microfluidic chip 100, the control operation condition of liquid treatment becomes a condition containing the heating setup temperature to perform heating treatment of liquid in a liquid treatment section. For example, in nucleic acid amplification reaction according to a PCR (Polymerase Chain Reaction) method, etc., temperature regulation of a reaction liquid of a mixture of template DNA, a primer, a substrate, a heat resistant polymerase enzyme, etc., is performed by liquid delivery control in the liquid channel and the reaction liquid is changed to predetermined three types of temperatures in sequence repeatedly, so that it is made possible to amplify the target DNA In the embodiment, the blood and the pretreatment reagent pass through the portion, whereby two strands of DNA extracted from leucocytes by the pretreatment reagent become one strand. To heat the heated section B uniformly, the channel substrate 21 is provided with the excavation 29 and this portion is thinned to about 1.2 mm.

The reagent converging section C makes the reaction amplification reagent converge into the blood and the pretreatment reagent subjected to the heating treatment. The magnitude relation of capillary forces of channels in the second port PT-D is port D exit channel 45>main channel 47>port D channel (second port PT-D) and a Laplace pressure valve is formed in the connection part of the port D exit channel 45 and the main channel 47. The reaction amplification reagent input to the second port PT-D remains on the connection face of the port D exit channel 45 and the main channel 47 without flowing out to the main channel 47. When the mixed liquid of the blood and the pretreatment reagent arrives at the port D exit channel 45 as operation described later is performed, the Laplace pressure valve is destroyed and the two liquids mentioned above converge.

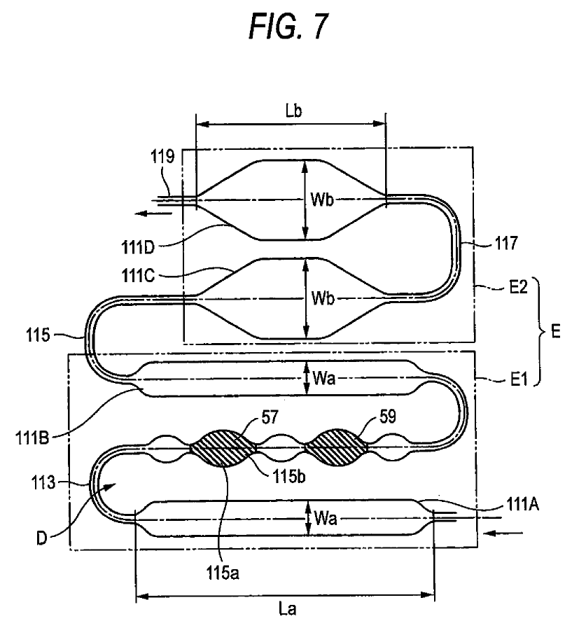

The enzyme mixing section E has a first mixing section E1 and a second mixing section E2 placed in order from the second port D as shown in FIGS. 4 and 7.

The first mixing section El is formed alternately with first channel parts 111A and 111B each with the perpendicular cross-section area in the flow direction of liquid being larger than the perpendicular cross-sectional area in any other channel and second channel parts 113 and 115 each having a smaller perpendicular cross-sectional area than the first channel part 111A, 111B. That is, the first channel part 111A at the preceding stage, the second channel part 113 at the preceding stage, the first channel part 111B at the following stage, and the second channel part 115 at the following stage are disposed in order from the upstream side.

The second mixing section E2 is formed alternately with first channel parts 111C and 111D each with the perpendicular cross-section area in the flow direction of liquid being larger than the perpendicular cross-sectional area in any other channel and second channel parts 117 and 119 each having a smaller perpendicular cross-sectional area than the first channel part 111C, 111D. That is, the first channel part 111C at the preceding stage, the second channel part 117 at the preceding stage, the first channel part 111D at the following stage, and the second channel part 119 at the following stage are disposed in order from the upstream side.

The perpendicular cross-sectional area of the first channel part 111A, 111B in the first mixing section El is formed smaller than that of the first channel part 111C, 111D in the second mixing section E2. In the embodiment, the depths of the mixing sections (vertical direction depth to the plane of the drawing of FIG. 4) are made the same and width Wa of the first channel part 111A, 111B is formed smaller than width Wb of the first channel part 11IC, 111D (Wₐ<W_{b}) as shown in FIG. 7. Channel direction length Lₐ of the first channel part 111A, 111B in the first mixing section E1 is formed longer than channel direction length L_{b}. Of the first channel part 111C, 111D in the second mixing section E2 (Lₐ>L_{b}). In the embodiment, the first channel parts 111A, 111B, 111C, and 111D are formed in parallel with each other and the second channel parts 113, 115, 117, and 119 are formed so as to join the first channel parts, but the placement is not limited to it and any desired placement may be adopted.

Thus, the enzyme mixing section E according to the embodiment is provided with the first mixing section El at the preceding stage of the second mixing section E2. The first mixing section E1 is formed like an elongated shape, whereby when a plurality of types of liquids different in wettability are stored in the channel in an unmixed state, if a liquid component having high wettability is deposited on the channel face and remains, deviating of the meniscus curved surface liquid end formed because of the wettability difference from the channel center is decreased although described later in detail. Accordingly, occurrence of an air bubble in the mixing section can be prevented.

That is, according to the configuration, a plurality of types of liquids are preliminarily mixed in the first mixing section E1 wherein a difference is hard to occur in the proceeding degree of the meniscus curved surface liquid end. This suppresses the difference in the proceeding degree of the meniscus curved surface liquid end caused by the fact that liquids different in wettability come in contact with the channel face in the second mixing section E2 high in mixing performance.

Preferably, the volume or each of the first channel part 111A at the preceding stage and the first channel part 111B at the following stage is set to a volume capable of storing the whole liquid once delivered from the second port PT-D and preferably is 80% or more of the volume of the whole delivered liquid. Accordingly, after the whole liquid is stored in the First channel part 111A at the preceding stage in the first mixing section E1, the liquid passes through the second channel part 113 at the preceding stage and is stored in the first channel part 111B at the following stage and passes through the wide channel parts and the narrow channel parts alternately, whereby agitation of orifice effect is performed more than once and mixing a plurality of types of liquids can be promoted.

The enzyme retention section D is placed in the second channel part 113 between the first channel parts 111A and 111B Like the mixing section A, the enzyme retention section D is implemented as a channel formed alternately with wide channel parts 115A and narrow channel parts 115B in the flow direction of liquid, some of the wide channel parts 115A become a reagent retention cell for retaining a reagent 57 dried and solidified by freezing and drying after a water solution of polymerase and dextrin is put as a drip and a reagent 59 dried and solidified by freezing and drying after a water solution of MutS and dextrin is put as a drip.

The enzyme mixing section E causes a converged liquid of blood, pretreatment reagent, and reaction amplification reagent to go and return between the first channel parts 111A and 111D of the first mixing section E1, thereby dissolving the reagent 57 of a first enzyme and the reagent 59 of a second enzyme and mixing the converged liquid.

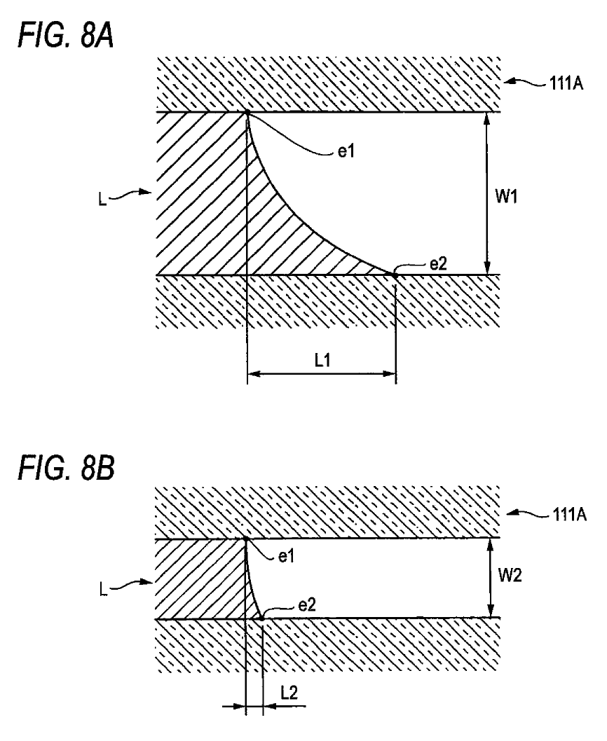

FIGS. 8A and 8B show a state in which a comparison is made between the case (a) where the width of the first channel part (cross-sectional area) is large and the case (b) where the width is small, respectively. How the liquid in the first channel part 111A (as well as 11B) proceeds varies from one place to another because the channel face is different wettability. That is, for width W1 shown in FIG. 8A, as the meniscus curved surface liquid end of liquid L, one end is e1 and an opposite end is e2 proceeding by distance L1 from e1 and the difference between the ends e1 and e2 appears as large deviation of the meniscus Curved surface liquid end. In contrast, for width W2 shown in FIG. 8B, since the width of the first channel part 111A is small, if the channel face is different wettability, occurring deviation of the meniscus curved surface liquid end lessens. That is, the difference between the ends e1 and e2 becomes short in proportional to the channel width and distance L2 becomes short. Consequently, stable preliminary mixing can be performed without occurrence of an air bubble in the first channel part and without fruitless overflowing of liquid from the first channel part.

This means that the first channel part 111A, 111B of the first mixing section E1 is formed finely to such an extant that the meniscus formed by the liquid end of liquid in the channel part becomes roughly symmetrical with respect to an axis center 51 of the first channel part 111A, 111B. The expression “roughly symmetrical” refers to an extent that the difference between the ends e1 and e2 of the meniscus becomes a quarter or less relative to the channel width. Preferably, the relationship between the width Wₐ of the first channel part 111A, 111B and the width W_{b} of the first channel part 111C, 111D is set to Wₐ=W_{b}/2.

The meniscus refers to a curved surface produced as the center of liquid in a narrow channel swells or sinks as compared with the portion along the surface in the channel; it occurs due to a capillary phenomenon. The capillary phenomenon is a phenomenon in which liquid in a fine channel attempts to flow along the channel; the degree is proportional to the surface tension of the liquid and is inversely proportional to the cross-sectional area of the channel. The surface tension in a force with which the surface of liquid shrinks and attempts to take a small area as much as possible; it acts along the surface.

In the microfluidic chip 100, if liquids different in wettability are mixed as in the embodiment, the liquids are preliminarily mixed in the first mixing section E1, whereby the difference in the proceeding degree of the meniscus curved surface liquid end is suppressed in the second mixing section E2 high in mixing performance at the following stage. Particularly, if the liquids are blood and a diluent, the blood and the diluent are reliably preliminarily mixed in the first mixing section E1 and accordingly the difference in the proceeding degree of the meniscus curved surface liquid end is suppressed in the second mixing function E2 high in mixing performance and occurrence of a liquid unfilled part is prevented and uniform dilution of the blood is made possible.

In the example shown in the figure, each of the mixing sections E1 and E2 is provided with two first channel parts, but the number of the first channel parts is not limited to it and an additional first channel part may be formed alternately with the second channel part.

The channels upstream and downstream from the wide channel part 115A of the enzyme retention section D retaining the reagents 57, 59 are thinner than the retention section and if there is no adhesion of the dried and solidified reagents 57, 59 to the channel, the solidified reagents 57, 59 are prevented from peeling off and flowing out to the preceding or following channel due to vibration of retention, transport, etc., of the chip 100.

Polymerase of the reagent 57 may be polymerase having strand displacement activity {strand displacement capability} and any polymerase of normal temperature property, moderate temperature property, or heat resistance property can be used preferably. Polymerase may be a natural body or may be a variant provided by artificially varying the natural body. As such polymerase, DNA polymerase can be named. Further, preferably the DNA polymerase has substantially no 5′->3′ exonuclease activity. As such DNA polymerase, a variant losing 5′->3′ exonuclease activity of DNA polymerase derived from thermophile Bacillus bacteria such as Bacillus steazothermophilus (which will be hereinafter called D. st) or Bacillus caldotenax (which will be hereinafter called B. ca), Klenow fragment of DNA polymerase I derived from Escherichia coli (E. coli), etc., can be named.

Dextrin is used as an enzyme stabilizing agent, whereby it is made possible to preserve enzymes for a long period of time and the enzyme in reaction liquid is also stabilized in amplification reaction and thus it is made possible to increase the amplification efficiency of nucleic acid. As other enzyme stabilizing agents, glycerol, bovine serum albumin, saccharides, etc., can be used.

The reagent 59 is placed downstream from the reagent 57 and is a reagent dried and solidified by freezing and drying after a water solution of MutS and dextrin is put as a drip. MutS is one of protein groups called “mismatch binding protein” (also called ”mismatch recognition protein”). When a partial mismatch base pair in two strands of DNA occurs, MutS is a protein group having a function of recovering it. In addition to the MutS protein (International Patent Publication No. 9-504699), various mismatch binding proteins such as MutM protein (JPA-2000-00265) are known.

The enzyme mixing section E causes the converged liquid of the blood, the pretreatment liquid, and the reaction amplification reagent to go and return between the first mixing section 49 and the second mixing section 51, dissolves the reagents 57 and 59, and preliminarily mixes the converged liquid At the same timer the enzyme mixing section E eliminates air bubbles in the channel. Further, it causes the converged liquid to go and return between the first channel parts 111C and 111D of the second mixing section E2, thereby essentially mixing the converged liquid and mixing the liquid more uniformly. To stably transport so that a droplet does not involve an air bubble at the going and returning time, it is desirable that the enzyme mixing section E should be water repellent for the mixed liquid; in the embodiment, COP (contact angle of water is about 110°) is selected is the material of the channel substrate 21.

In the reaction section F, a water solution of a primer and gelatin of the target DNA is put as a drip and then is cooled, solidified, and fixed. The primer is oligonucleotide of about 20 base length having a complementary base sequence in a specific portion of the target DNA and becomes a staring point of DNA synthesis by polymerase. In the embodiment, 13 reaction detection cells 27a to 27m are formed and to perform amplification reaction specifically for sequence of wild and mutant for the gene to be tested, a primer 61 for amplifying wild and a primer 63 for amplifying mutant are paired and are fixed to different reaction detection cells.

That is, genes at six places D1 to D6 are to be tested in the 12 reaction detection cells 27a to 27l. A primer 65 for amplifying a gene sequence where polymorphism does not exist is fixed in the reaction detection cell 27m at the remaining PD and this cell is used as positive control. The sample mixed in the first mixing section 49 and the second mixing section 51 is dispensed to the reaction detection cells 27a to 27m in a fixed quantity.

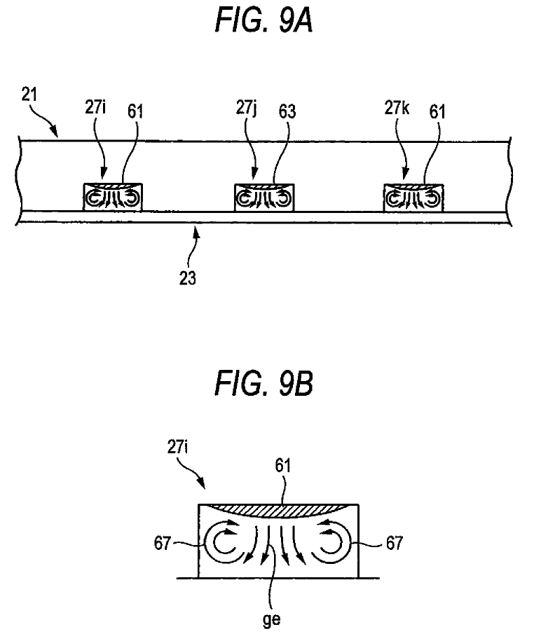

FIG. 9A is a sectional view taken on line P2-P2 in FIG. 4 where primers are mixed and diffused and FIG. 9B is a schematic representation of the reaction detection cell as a main part enlarged view.

The reaction detection cells 27a to 27m are heated to 60° C., whereby solidified gelatin dissolves and is dispersed in the reaction detection cells 27a to 27m and isothermal amplification reaction is performed. Only a water solution of primer can be put as a drip on the reaction detection cells 27 and be dried and solidified. In this case, however, when liquid flows into the cell, the primer is allowed to flow in the flow direction and reaction, detection in the cell cannot be executed. Thus, gelatin hard to dissolve in a normal-temperature water solution is contained 0.5% and is put as a drip and is solidified.

The water solution of the primer and gelatin is put as a drip and is fixed to the cell on the side of the channel substrate 21 and is placed on the upper face of the channel as shown in FIGS. 9A and 9B in a microchip use state. After liquid flows in, it is heated from the lid 23 side, namely, the lower face, whereby gelatin ge containing the primer 61 dissolved with temperature rise of the liquid flows to the lower side in the channel by gravity because the specific gravity of the gelatin is large. The liquid is heated from the lower face, thereby causing convection 67 to occur in the cell. The primer 61 and the gelatin ge are mixed and diffused uniformly in a short time in the reaction detection cells 27 because of the multiplier effect of the flow to the lower side of the channel caused by the gravity of the gelatin ge and the convection 67 caused by heating the liquid.

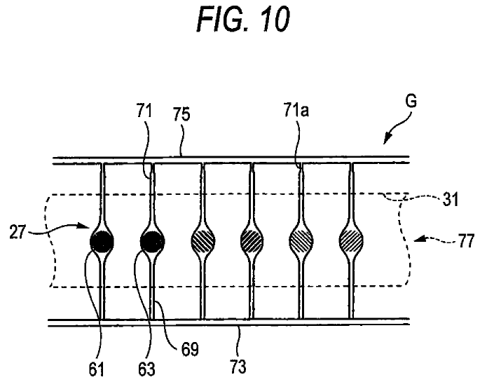

FIG. 10 is an enlarged plan view of the reaction detection cells 27.

A reaction detection cell entrance channel 69 and a reaction detection cell exit channel 71 are placed before and after each of the reaction detection cells 27a to 27m and each of the entrance and exit channels 69 and 71 is a narrow channel. The end face of the liquid after dispensing remains on the connection face of the entrance channel 69 and a main channel 73 and on the connection face of the exit channel 71 and an exhaust channel 75. The reaction section F is formed with a heating section 77 and for the heating section 77 to heat uniformly, the channel substrate 21 is thinned to about 1.2 mm in the presence of the excavation 31 mentioned above. The heating section 77 is placed so as to heat the whole reaction detection cells 27 and parts of the entrance and exit channels 69 and 71, and the temperature of any other portion than the heating section 77 is regulated by another temperature regulation unit. That is, both end faces of the liquid in each reaction detection cell 27 are kept at the normal temperature without being heated. Accordingly, heating can be prevented from evaporating water. The heating section 77 and the temperature regulation unit on the periphery thereof make up the heat regulation section 15 in FIG. 1.

The entrance and exit channels 69 and 71, the main channel 73, and the exhaust channel 75 make up the fixed-quantity dispensing channel G. The fixed-quantity dispensing channel G dispenses a fixed quantity of the second mixed liquid treated in the enzyme mixing section E to each of a plurality of the reaction detection cells 27 of the reaction section F.

The liquid dispensed in a fixed quantity to the reaction detection cells 27 contains the sample liquid having biological cells, the pretreatment reagent, and the reaction amplification reagent. The primers 61, 63, . . . of pieces or fragments of nucleic acid are installed in each reaction detection cell 27 and a fixed quantity of liquid is dispensed to the reaction detection cell 27 and while the liquid is heated, excitation light is applied, whereby fluorescence occurring in the liquid treatment section is detected. Nucleic acid amplification reaction of the detected substance is Conducted in the reaction detection cell 27. At this time, a labeled substance having a specific interaction carrying a photogenic substance of a label substance having high detection sensitivity, for example, a labeled antibody, a labeled antigens a labeled nucleic acid, or the like is used. Cybergreen is intercalated into double stranded DNA amplified by reaction, whereby it emits strong fluorescence. The fluorescence strength is measured, whereby it is made possible to detect the presence or absence of a gene sequence as a target.

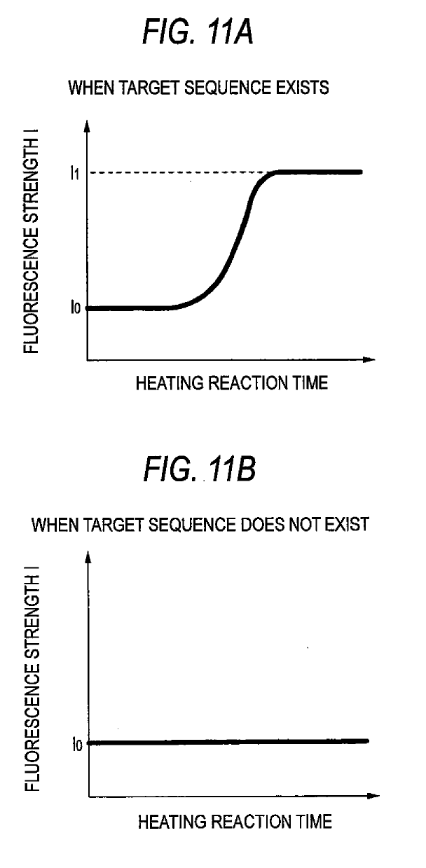

FIG. 11A is a graph of the fluorescence measurement result when a target sequence exists and FIG. 11B is a graph of the fluorescence measurement result when a target sequence does not exist.

Each of the reaction detection cells 27a to 27m is excited at a wavelength of about 490 nm by an optical system and fluorescence of about 520 nm of intercalated cybergreen is measured, whereby amplification of the target DNA is recognized. That is, as shown in FIG. 11A, if a nucleic acid sequence as a target exists, an increase in fluorescence strength I is recognized; if a nucleic acid sequence as a target does not exist, an increase in fluorescence strength I is not recognized.

In the reaction section F, to make it possible to allow liquid to smoothly enter at the dispensing time to the reaction detection cells 27a to 27m and stably stop with the Laplace pressure valve at the exit, it is desirable that the reaction detection cells 27 and the narrow entrance and exit channels 69 and 71 placed before and after the reaction detection cells 27a to 27m should be hydrophilic in moderation. In the embodiment, at least the entrance and exit channels 69 and 71 are made hydrophilic by plasma irradiation (contact angle of water is about 70°).

As a method of making the channel substrate 21 partially hydrophilic or water-repellent, a known method (a method of applying hydrophilic/water repellent treatment liquid, a method of forming a thin film of hydrophilic/water repellent material by UV irradiation, vapor deposition, or sputtering, a method of molding using resins different in wettability by two-color molding or insert molding, or the like) can be used in addition to plasma irradiation. In the embodiment, the inner faces in the channels (at least the entrance and exit channels 69 and 71) have wettability of at least two levels or more. Accordingly, it is made possible to allow liquid to smoothly enter at the dispensing time to the reaction detection cells 27a to 27m and stably stop with the Laplace pressure valve at the exit.

Here, foaming in the reaction detection section will be discussed.

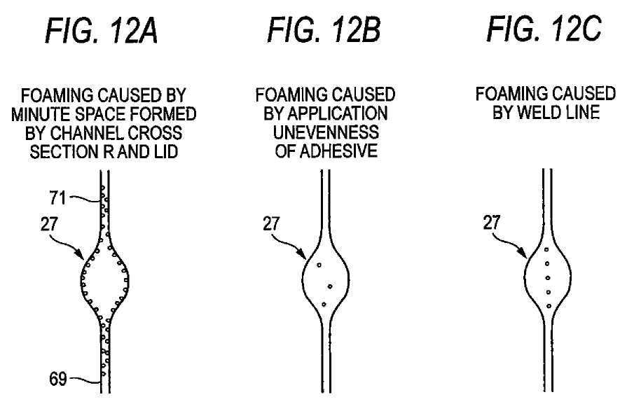

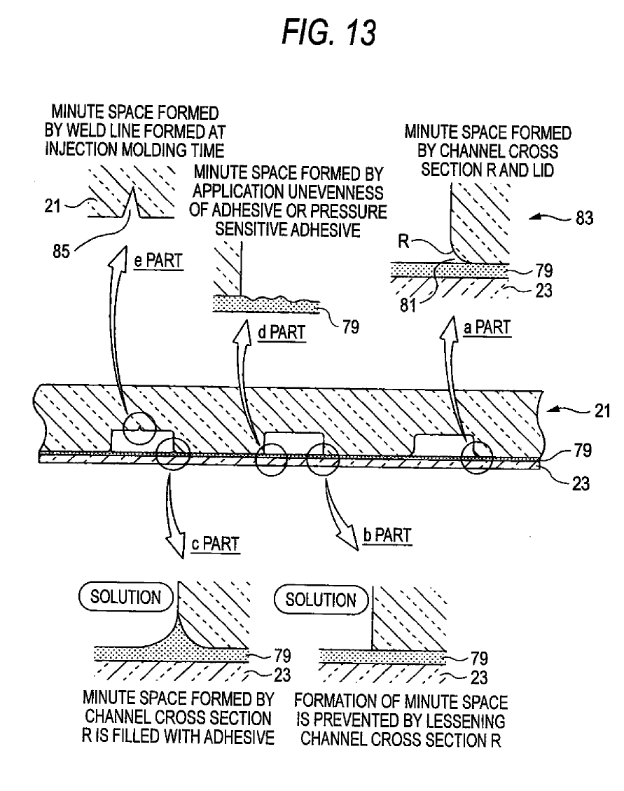

FIGS. 12A to C are plan views to represent the foaming situation of the reaction detection section and FIG. 13 is main part sectional views to represent foaming prevention measures of the reaction detection section.

When the reaction detection cells 27a to 27m are heated, if an air bubble occurs in the reaction detection cell 27, accuracy of fluorescence detection is degraded; this is a problem. Thus, it is necessary to prevent air bubble occurrence in the channel. The air bubble occurrence mechanism is as follows: As shown in FIGS. 12A to C, when mixed liquid flows into the reaction detection cell 27, if a minute space is formed in the reaction detection c:ell 27 for some cause of channel cross section R (chamfer radius of channel corner), adhesive application unevenness, a weld line, etc., the mixed liquid cannot flow into the minute space and minute wet residue, namely, a minute air pocket occurs. As the air pocket expands and grows by heating, an air bubble occurs.

As a representative example of the minute space, a minute space 81 formed by a joint part of channel cross section R and the lid 23 shown in a part of FIG. 13 can be named. As prevention measures, it is effective to lessen the cross section R of a channel 83 as much as possible (preferably 100 μm or less, more preferably 10 μm or less) as shown in b part of FIG. 13.

As another measure, a method of filling the minute space 81 formed by the cross section R of the channel 83 and the lid 23 with an adhesive 79 by optimizing the application condition or putting condition of the adhesive 79 as shown in c part of FIG. 13 is also available.

As another example of the minute space 81, the adhesive 79 of the lid 23 or application unevenness of the adhesive 79 exists as shown in d part of FIG. 13. If the minute space 81 is formed because of application unevenness of the adhesive face, the minute space S1 is formed as with the channel cross section R, causing an air bubble to occur. As another example, a weld line 85 of the channel substrate 21 manufactured by injection molding exists as shown in e part of FIG. 13; if the weld line 85 forms a similar minute space 81, an air bubble is caused to occur. Thus, preferably, particularly the inner face of each channel of the reaction section F is a continuous smooth face for preventing formation of a minute gap space not filled with liquid when the liquid flows through the inside of the channel. Accordingly, an air bubble is prevented from occurring in the channel at the heating time and degradation of the fluorescence detection accuracy can be prevented. Thus, to prevent an air bubble from occurring, it becomes necessary to prevent formation of a minute space 81 into which liquid can flow when the liquid flows in by selecting any appropriate one of the solution measures described above.

In the embodiment, the 12 reaction detection cells 27a to 27l for determining single nucleotide polymorphism of six sets and one reaction detection cell 27m for positive control are provided as mentioned above. The primer 65 with a gene sequence where polymorphism does not exist as a target is installed in the reaction detection cell 27m and if any sample is tested, growing of the fluorescence strength can be recognized. The fluorescence of the reaction detection cell 27m for positive control is recognized, whereby it can be checked that the liquid delivery operation sequence has been performed normally and normal reaction has been conducted, and it is made possible to guarantee the reliability of the testing result.

As a guaranteeing method of negative control, it may be checked that the fluorescence strength does not grow by inputting water rather than blood and conducting the reaction sequence or two circuits may be formed on the same substrate for performing testing and guaranteeing of negative control at the same time.

To operate limited liquid with the microfluidic chip 100, particularly to perform complicated handling of liquid by pneumatic drive from the outside of the chip with the microfluidic chip 100 formed of simple channels not containing any active valve or pump, it is indispensable to precisely detect the position of the liquid. For example, if an attempt is made to control according to the flow quantity of drive air without detecting the position of the liquid, it becomes difficult to handle the liquid with good reproducibility because of disturbance of expansion or shrinkage of the air volume (dead volume) of piping from the pump to the port section PT of the microfluidic chip 100 or the channel in the chip, caused by temperature change, change in flow quantity resistance caused by a minute flaw or static electricity in the channel, the effect of vapor pressure caused by evaporation of fluid when the fluid is heated, or the like. Thus, it becomes very difficult to precisely detect the position of the liquid.

The microfluidic chip 100 detects at least ether the leading end or the trailing end of liquid in the liquid channel at a specific position of the liquid channel and determines the control operation condition of the liquid in response to the end detection timing. Accordingly, the need for intricate operation such as operation of pipetting, taking out from, taking to the devices, etc., is eliminated and extraction or reaction of amplification, etc., of DNA in the sample in the liquid channel is made possible. If the control operation condition of the liquid contains at least one of the liquid move direction, the liquid move speed, and the drive force for liquid move, move control of delivered liquid in the liquid channel is made possible. According to a configuration wherein all of the liquid move direction, the liquid move speed, and the drive force can be controlled, the operation conditions are switched as desired, whereby liquid delivery control equivalent to performing operation of pipetting, taking out from, taking to the device, etc., is made possible.

In the embodiment, the position of the liquid is detected, whereby control of the liquid drive speed, the liquid drive direction, and the drive force is switched by the control section 19 of the testing apparatus 11. It is assumed that the drive force contains the atmospheric release state and the closed sate of the port section PT and the joint state of a plurality of port sections PT in addition to suction and pressurization under given pressure.

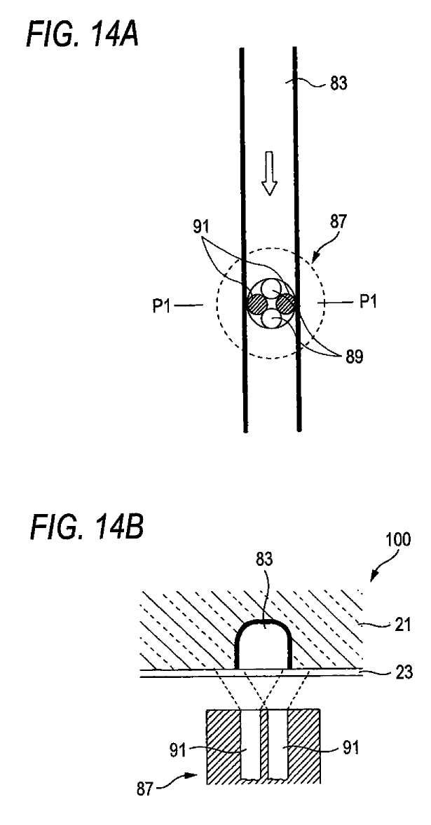

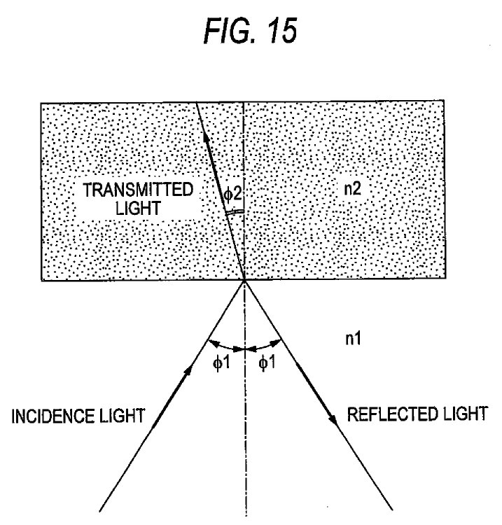

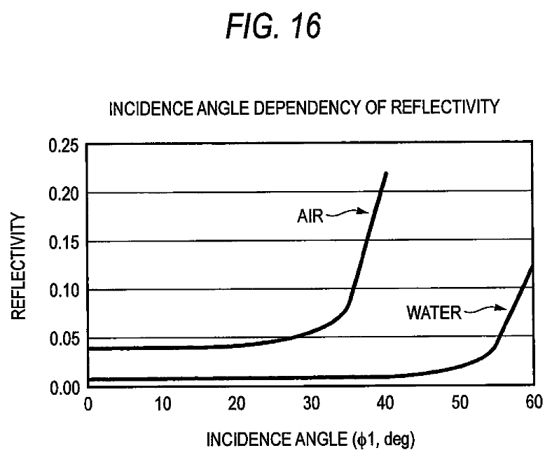

FIG. 14A is a plan view of the liquid position detection section and FIG. 14B is a sectional view taken on line P1-P1 in FIG. 14A, FIG. 15 is a schematic drawing to represent incidence light and reflected light of the liquid position detection section, and FIG. 16 is a graph to represent the correlation between reflectivity and incidence angle.

Sensing sections PH1 to PH5 for detecting the liquid position (see FIGS. 1 and 4) are placed in the microfluidic chip 100. In the embodiment, the liquid position detection sections 16 are placed at the positions opposed to the sensing sections PH1 to PH5. Although one liquid position detection section 16 is shown collectively in FIG. 1, the liquid position detection sections 16 are placed opposed to the sensing sections PH1 to PH5 in a one-to-one correspondence with each other. As a specific example of the liquid position detection section 16, a reflection optical fiber sensor 87 shown in FIG. 12 is used. The tip of each optical fiber sensor 87 is placed toward the channel 83 from the lid 23 side of the chip 100, as shown in FIG. 145.

The reflection optical fiber sensor 87 irradiates a specific position of the channel 83 with light, detects reflected light from the channel 83, and determines the presence or absence of liquid in the channel at the specific position from light amount change based on reflectivity change of the reflected light between air and liquid. Therefore, it is made possible to irradiate with light from the outside of the chip 100 and determine the presence or absence of liquid by reflectivity change of the reflected light, so that the sensor, etc., is not exposed to the channel 83 and contamination of sample liquid does not occur. Vibration occurring if supersonic waves are used does not occur and mutations in the mix degree with the reaction liquid, etc., do not occur.

Specifically, the reflection optical fiber sensor 87 supplies light to irradiate a specific position through a light emission optical fiber 89 and introduces reflected light from the channel 83 into a light reception optical fiber 91 for detection. According to the reflection optical fiber sensor 87, light irradiation and light reflection can be performed on the fiber tip face of a small area into which the light emission optical fiber 89 and the light reception optical fiber 91 are integrated, it is made possible to irradiate the small detection area with light and receive the reflected light from the area, and it is made possible to detect the presence or absence of liquid at the specific position of the minute channel 83.

The light reception optical fiber 91 of the reflection optical fiber sensor 87 detects the strength of reflected light from the chip 100.

The presence or absence of a droplet in the channel 83 can be detected mainly based on the difference between the reflectivity from the channel Side of the lid 23 when air exists in the channel and that when water exists in the channel. Generally, the reflectivity relative to incidence light as shown in FIG. 15 is represented by the following expression:

$$Rp = {( \frac{{n}^{2} cos φ 1 - \sqrt{{n}^{2} - {sin}^{2} φ 1}}{{n}^{2} cos φ 1 + \sqrt{{n}^{2} - {sin}^{2} φ 1}} )}^{2}$$

$$Rs = {( \frac{cos φ1 - \sqrt{{n}^{2} - {sin}^{2} φ1}}{cos φ1 + \sqrt{{n}^{2} - {sin}^{2} φ1}} )}^{2}$$

where Rₚ: p polarization, Rₐ: s polarization, and n=n2/n1. If

$$\overline{R} = \frac{Rp + Rs}{2}$$

is set and the reflectivity of the lid 23 is set to n1=1.49 and the reflectivity of fluid in the channel 83 is set to

n2 (air)=1.00 when no droplet exists

n2 (water)=1.33 when a droplet exists,

calculation is performed, whereby the difference between the reflectivity when the fluid in the channel 83 is air and the reflectivity when the fluid in the channel 83 is water can be found as in FIG. 16.

If the spread angle of light emission of the used light emission optical fiber 89 is 60°, the range of 0° to 30° in FIG. 16 may be considered; if the fluid in the channel 83 is air, the reflectivity becomes about 4% and if the fluid in the channel 83 is water, the reflectivity becomes 0.5% or less. According to the difference, the light reception amount of the reflection optical fiber sensor 87 changes based on the presence or absence of a droplet and droplet arrival can be detected.

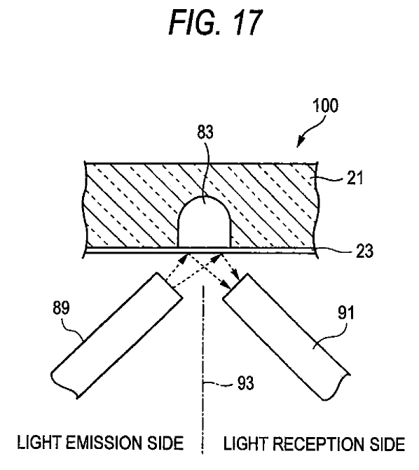

As seen in FIG. 16, as the incidence angle becomes larger, the reflectivity difference between air and water becomes larger, so that the reflection optical fiber sensor 87 has the light emission optical fiber 89 and the light reception optical fiber 91 as a separation type as in FIG. 17, and can be placed at an angle relative to the chip 100. FIG. 17 is a side view of the liquid position detection section wherein the light emission optical fiber and the light reception optical fiber are placed as they are inclined. In the configuration in the figure, the direction in which the specific position of the channel 83 is irradiated with light and the detection direction of reflected light from the specific position are set to inclined directions relative to a normal 93 to the light irradiation face of the specific position According to such a configuration, at the inclination angle, when air exists, reflected light is placed out of the light reception optical fiber 91 and when liquid exists, reflected light is made incident on the light reception optical fiber 91, so that detection of liquid based on the reflectivity change can be conducted more stably in the simple structure. The fiber diameters, the emission light and incidence light angles, the placement, the used number, etc., of the light emission optical fiber and the light reception optical fiber can be optimized experimentally or by optical simulation in response to the detection channel shape.

Thus, detection of the reflection optical fiber sensor 87 is detection of the reflectivity difference between air and fluid; it has the advantage that it the type or the density of a dissolved substance in fluid changes, stable detection can be conducted as compared with a detection method based on the principle of detecting dispersion of light.

The microfluidic chip 100 according to the embodiment having the configuration described above includes:
(1) the first port PT-A for inputting sample liquid and a pretreatment reagent;
(2) the second port PT-D for inputting a reaction amplification reagent;
(3) the third port PT-B for supplying air pressure to the channel;
(4) the sample mixing section A for mixing the sample liquid and the pretreatment reagent input from the first port PT-A to generate first mixed liquid;
(5) the heated section B for heating the first mixed liquid, extracting DNA from the biological cell, and decomposing the DNA into a single strand;
(6) the reagent converging section C for allowing the reaction amplification reagent to converge into the first mixed liquid treated in the heated section B;
(7) the enzyme retention section D solidifying and installing an enzyme whose dissolution advances with the passage of the second mixed liquid converged in the reagent converging section C;
(8) the enzyme mixing section E for promoting mixing of the enzyme into the second mixed liquid treated in the enzyme retention section D;
(9) the reaction section F made up of a plurality of reaction detection cells 27 connected to the enzyme mixing section E for executing DNA amplification by dissolving and heating a primer solidified and installed in the channel and detection of DNA amplification at the same position; and
(10) the fixed-quantity dispensing channel G connected to the plurality of reaction detection cells 27 for dispensing a fixed quantity of the second mixed liquid treated in the enzyme mixing section E to each of the plurality of reaction detection cells 27. Thus, liquid delivery control can be performed according to the simple structure without requiring a stereoscopically complicated structure, the need for intricate operation of pipetting, taking out from, taking to the device, etc., is eliminated, and the precise and highly reliable analysis result can be provided at a low cost and in a short time by performing simple operation requiring no skill.

Next, a liquid delivery flow using the microfluidic chip 100 described above will be discussed.

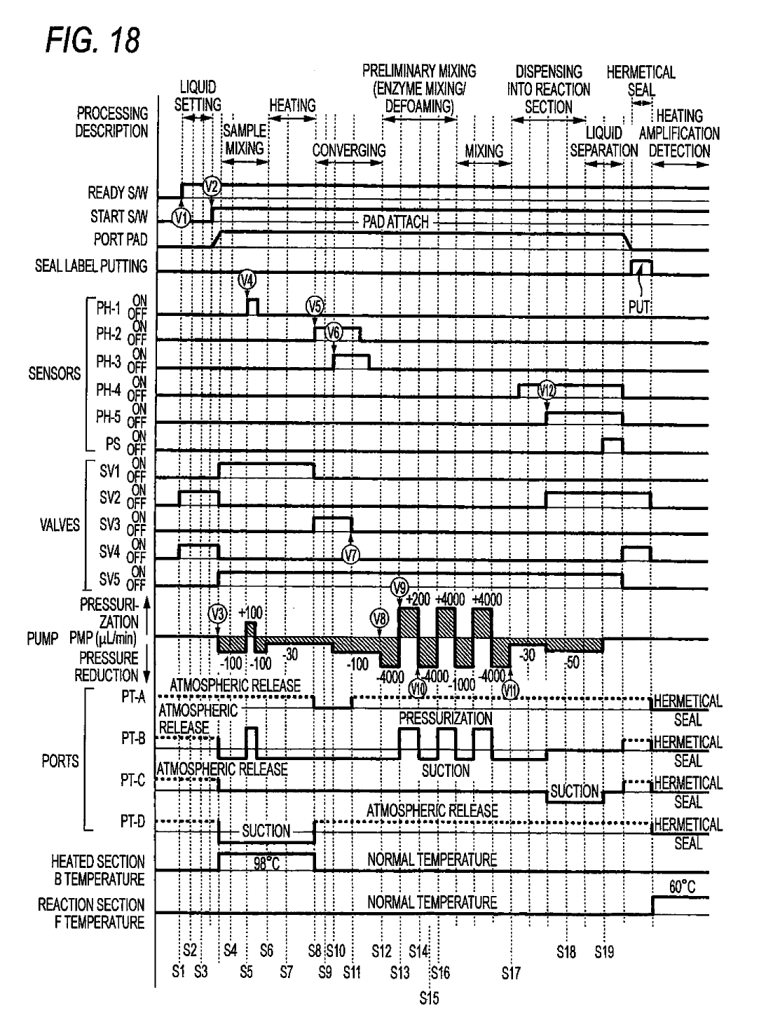

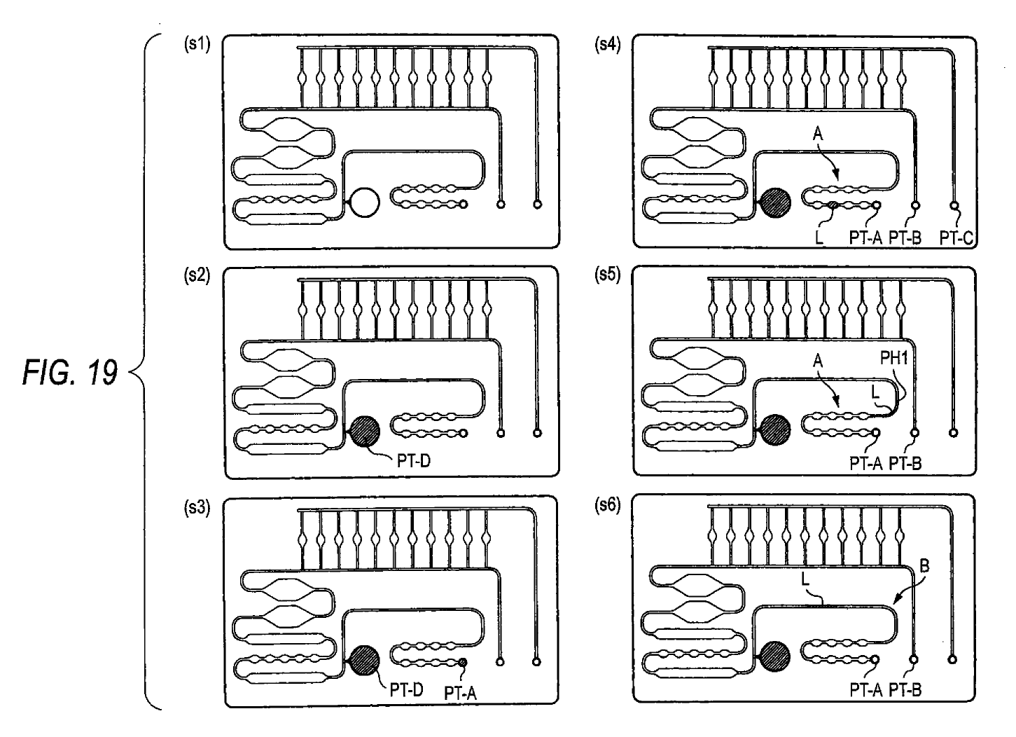

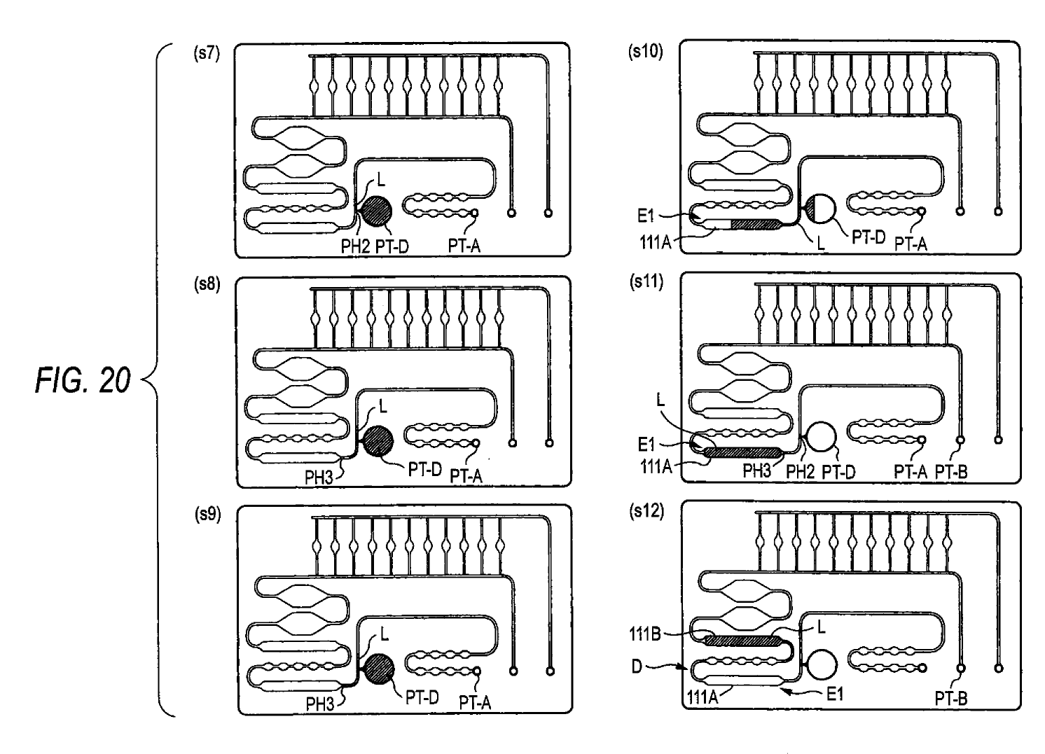

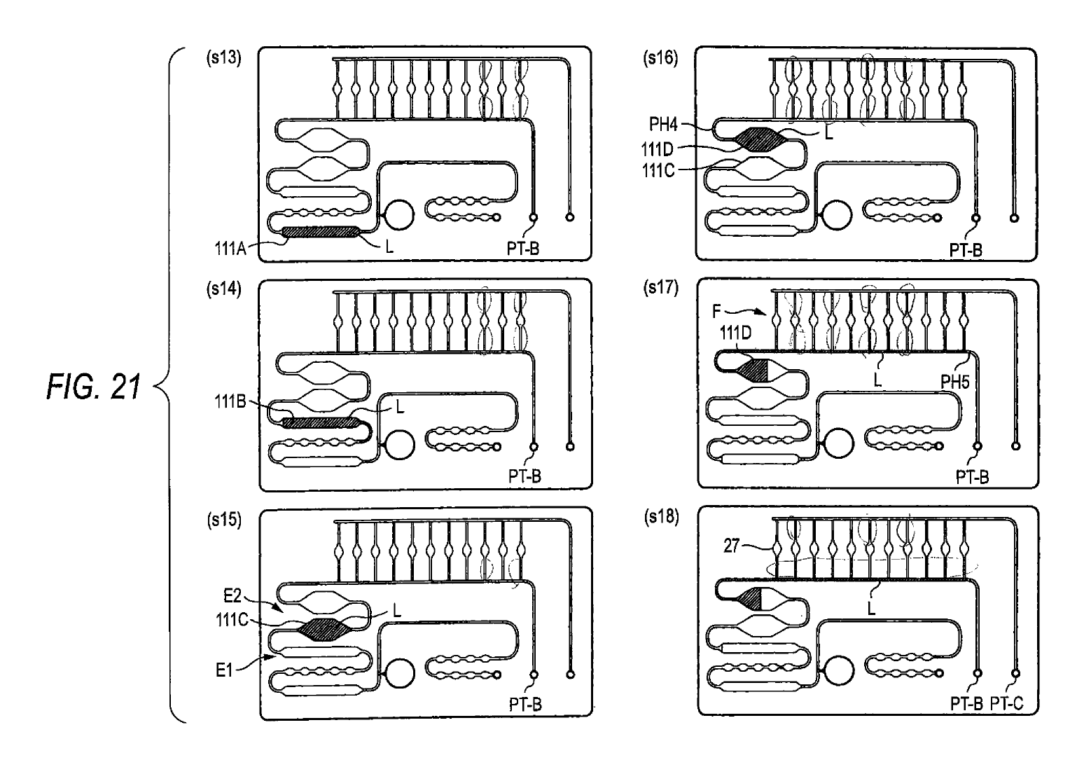

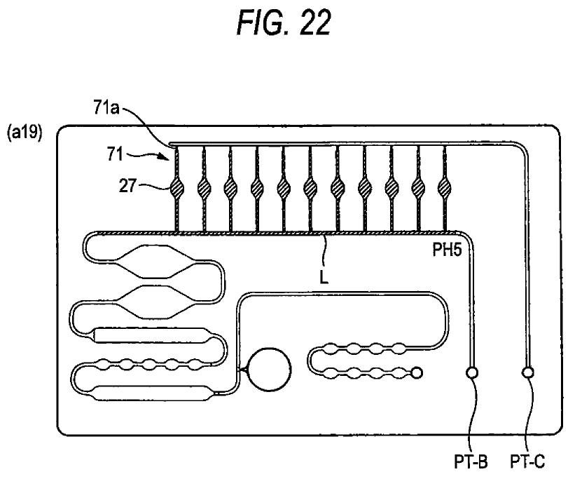

FIG. 18 is a time chart to represent the operation state of each component involved in the drive control of the microfluidic chip along the time axis, FIG. 19 is a schematic representation of the operation from liquid setting to the first heating, FIG. 20 in a schematic representation of the operation to enzyme mixing, FIG. 21 is a schematic representation of the operation to dispensing into the reaction sections and FIG. 22 is a schematic representation of the operation from dispensing to testing completion.

In the description to follow, control operation V1 to V3 in FIG. 18 and steps S1 to S19 in FIGS. 19 to 22 are associated with each other.

First, the chip 100 is prepared and a READY switch of the testing apparatus 11 is pressed (V1, S1). A reaction amplification reagent is input to the second port PT-D (S2). The magnitude relation of capillary forces of channels in the second port PT-D is port D exit channel 45>main channel 47>second port PT-D and a Laplace pressure valve is formed in the connection part of the port D exit channel 45 and the main channel 47. Thus, the reaction amplification reagent remains on the connection face of the port D exit channel 45 and the main channel 47 without flowing out to the main channel 47.

Next, blood and a pretreatment reagent are input to the first port PT-A (S3). The chip 100 is set in the testing apparatus 11 and a START switch of the testing apparatus 11 is pressed (V2). Then, port pads are pressed against the first port PT-A, the second port PT-D, the third port PT-B, and the fourth port PT-C. At this time, the pads corresponding to the first port PT-A, the second port PT-D, the third port PT-B, and the fourth port PT-C are placed in an atmospheric release state and as the pads are pressed against the ports, the liquid input to the chip does not move. Upon completion of pressing the pads against the ports, the pressure of the third port PT-B is reduced (V5) and the blood and the pretreatment reagent L pass through the sample mixing section A at high speed (100 μ/min), whereby they are mixed uniformly (S4). The second port PT-D is sucked as the same pressure reduction as the third port PT-B and if liquid delivery resistance of the blood and the pretreatment reagent is large, the pretreatment reagent in the second port PT-D does not flow out into the channel.

When the liquid arrives at the sensing position PH1 and a sensor PH-1 of the liquid position detection section detects the liquid and is turned ON (V4), the third port PT-B is pressurized and delivers a fixed quantity of liquid (10 μL) upstream in an opposite direction at high speed (100 μL/min) (S5). Then, the pressure of the third port PT-B is reduced (V3) and the third port PT-B and delivers a fixed quantity of liquid (10 μl) downstream at high speed (100 μL/min). As the reciprocating operation is performed, the liquid can be mixed more uniformly.

Next, the suction speed is switched to low speed (30 μL/min) (S6).

The liquid passes through the heated section B at low speed (30 μL/min) (S7), whereby the mixed liquid L of the blood and the pretreatment reagent is heated to 98° C. for a given time (for example, 15 seconds), and DNA in leucocytes is extracted, resulting in one strand.

When the liquid arrives at the sensing position PH2 and a sensor PH-2 is turned ON (V5), the second port PT-D is placed in an atmospheric release state and at the same timer the first port PT-A is closed and the reaction amplification reagent flows out from the second port PT-D into the main channel 47 by suction from the third port PT-B (S8). Accordingly, the mixed liquid L of the blood and the pretreatment reagent are converged without containing any bubble (S9).

When the liquid arrives at the sensing position PH3 and a sensor PH-3 is turned ON (V6), the suction speed is switched to high speed (100 μL/min) and a given flow quantity (45 μL) is sucked (S10).

The first port PT-A is placed in an atmospheric release state (V7), further 15 μL is sucked, whereby the second port PT-D becomes empty and the liquid is mixed in the first mixing section E1 (S11).

Further, 80 μL is sucked at higher speed (4000 μL/min) (V8), whereby the mixed liquid L passes through the first channel part 111A and the enzyme retention section D and the enzyme is dissolved and the liquid is mixed in the first channel part 111B (S12).

Further, 80 μL is pressurized at low speed (200 μL/min) (V9), whereby the mixed liquid L is returned to the first channel part 111A and minute air bubbles occurring at the enzyme dissolving time are slower than the liquid move speed and thus collect in the liquid rear end part and the air bubbles are brought away from the liquid with a move of the mixed liquid L, are deposited on the channel wall, burst, and disappear (S13).

Further, 80 μL is sucked at high speed (4000 μL/rain) (V10), whereby the liquid is mixed in the first channel part 111D (S14).

Similar reciprocating operation is also performed in the second mixing section E2 at the following stage, whereby the liquid is mixed uniformly. That is, the mixed liquid L is transported from the first channel part 111B of the first mixing section E1 to the first channel part 111C of the second mixing section E2 (S5) and further is sent to the first channel part 111D (S16) and is returned from the first channel part 111D to the first channel part 111C. The reciprocating operation of the mixed liquid L is also performed more than once in the second mixing section E2.

Next, suction is executed at low speed (30 μL/min) from the third port PT-B (V11), whereby the mixed liquid L in the first channel part 111D of the second mixing section E2 is transported to the channel of the reaction section F (S17).

When the liquid arrives at the sensing position PH5 and a sensor PH-5 is turned ON (V12), the third port PT-B is placed in a closed state, the fourth port PT-C is sucked at low speed (50 μL/min). The mixed liquid L is transported into the reaction detection cell 27 (S18) and stops at a small-diameter part 71a of the reaction detection cell exit channel 71 downstream from the cell (S19). At this stop timing, when the pressure sensor PS reaches a given pressure, it can be determined that dispensing to the reaction detection cell 27 is complete.

At this time, each reaction detection cell 27 is kept at the normal temperature and the primer previously immobilized with gelatin is retained in the cell without dissolving.

Next, the pad devices of the testing apparatus 11 are detached and labels Ra, Rb, Rc, and Rd (see FIG. 5) are put on the port sections PT-A, PT-B, PT-C, and PT-D with a seal device (not shown) and the chip 100 is placed in a hermetically sealed state, eliminating the fear of contaminating the environment as the amplification product resulting from amplification reaction flows out to the outside of the chip.

Next, the reaction section F is heated rapidly to 60° C. by a temperature regulation device (not shown). As it is heated, the primer solidified by gelatin diffuses uniformly in the reaction detection cell 27 and isothermal amplification reaction starts.

At this time, the liquid end faces of the narrow reaction detection cell entrance channel 69 and the narrow reaction detection cell exit channel 71 at both ends of the reaction detection cell 27 are not heated to 60° C. and are kept at the normal temperature and the liquid in the reaction detection cell 27 does not evaporate.

The reaction detection cells 27a to 27m are irradiated with excitation light in the fluorescence detection section 17 shown in FIG. 1 and fluorescence measurement is conducted at given time intervals, whereby whether or not the target gene sequence corresponding to the primer previously installed in each of the reaction detection cells 27a to 27m exists can be known. If the target gene sequence exists, it is recognized that the fluorescence strength grows; whereas, if the target gene sequence does not exist, the fluorescence strength does not grow.

Therefore, the microfluidic chip 100 according to the invention includes channels for mixing with various reagents and dispensing a fixed quantity of the mixed liquid as component measures in addition to the first port PT-A for inputting sample liquid and a pretreatment reagent, the second port PT-D for inputting a reaction amplification reagent, and the third port PT-D for supplying air pressure to the channel, detects at least either the leading edge or the trailing end of liquid in the liquid channel, and determines the control operation condition of the liquid in response to the end detection timing, whereby it is made possible to perform complicated handling of limited liquid by pneumatic drive from the outside of the chip 100 particularly with simple channels not containing any active valve or pump. This means that liquid delivery control is made possible according to a simple structure without requiring a stereoscopically complicated structure. Accordingly, simply by inputting a sample and a liquid reagent, automatically any desired droplet operation and chemical reaction are conducted and the need for intricate operation of pipetting, taking out from, taking to the device, etc., is eliminated and the high analysis result can be obtained.

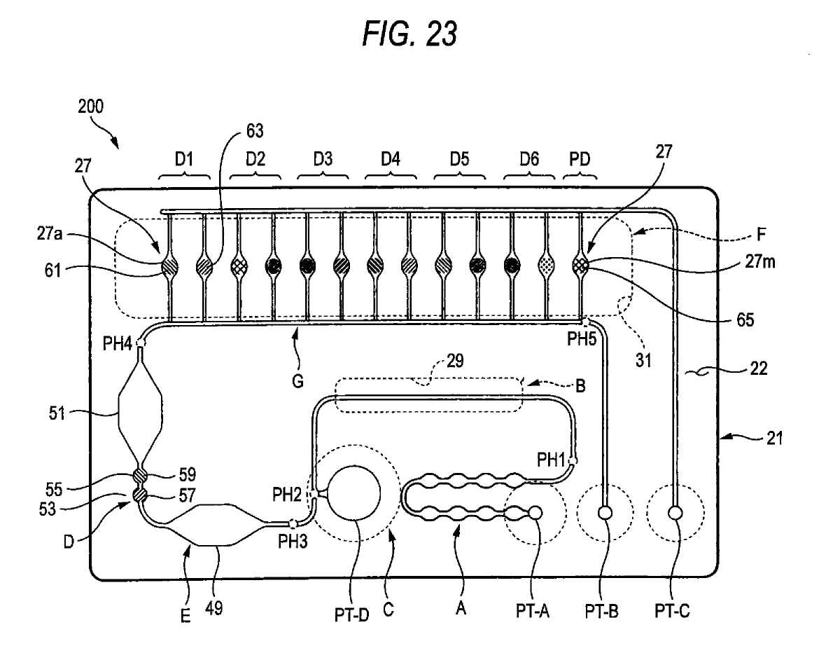

Next, another embodiment of a microfluidic chip according to the invention will be discussed FIG. 23 is a plan view to represent the bottom view of a microfluidic chip 200 in the embodiment of the invention. Parts identical with those previously described with reference to FIG. 4 are denoted by the same reference numerals in FIG. 23 and will not be discussed again.

The microfluidic chip 200 of the embodiment differs from the microfluidic chip of the embodiment described above in configurations of fourth channel (enzyme retention section) D and fifth channel (enzyme mixing section) E for promoting mixing of an enzyme into second mixed liquid treated in the enzyme retention section D.

The enzyme mixing section E has a first mixing section 49 of a liquid reservoir and a second mixing section 51, as shown in FIG. 23. The enzyme retention section D is provided between the first mixing section 49 and the second mixing section 51 and is made up of a first retention section 53 and a second retention section 55. The first retention section 53 is a reagent retention cell installed between the first mixing section 49 and the second mixing section 51 for retaining a reagent 57 dried and solidified by freezing and drying after a water solution of polymerase and dextrin is put as a drip.

The channels upstream and downstream from the retention section are thinner than the retention section and if there is no adhesion of the dried and solidified reagent 57 to the channel, the solidified reagent 57 is prevented from peeling off and flowing out to the preceding or following channel due to vibration of retention, transport, etc., of the chip 200.

Next, a liquid delivery flow using the microfluidic chip 200 described above will he discussed.

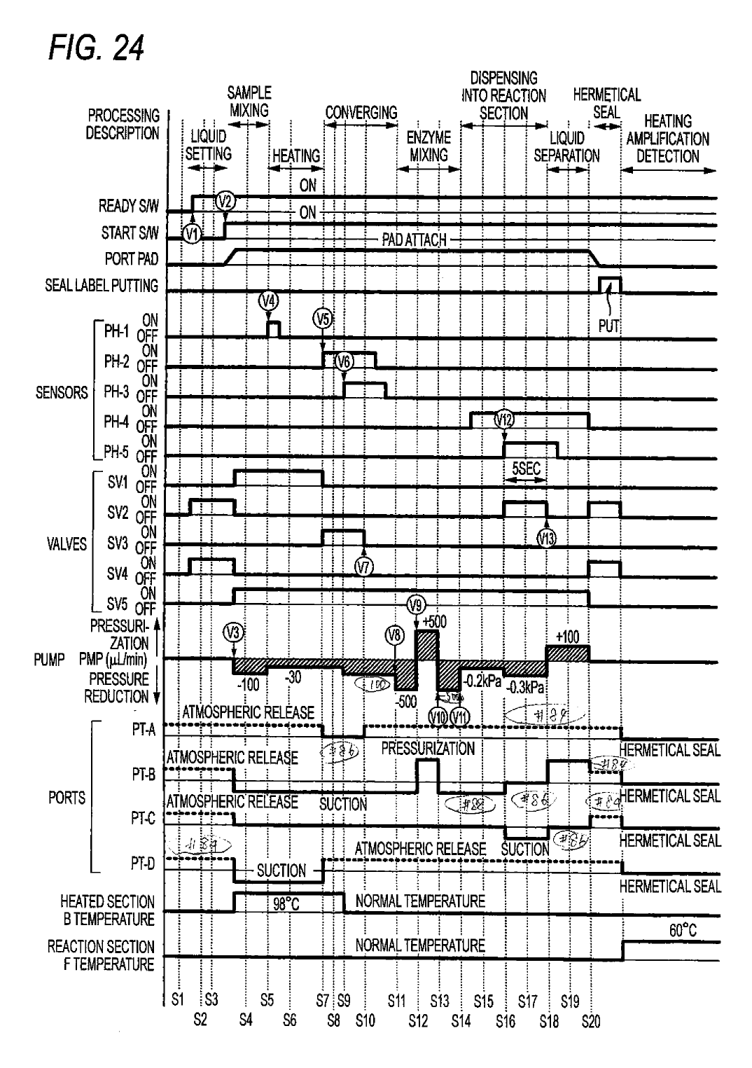

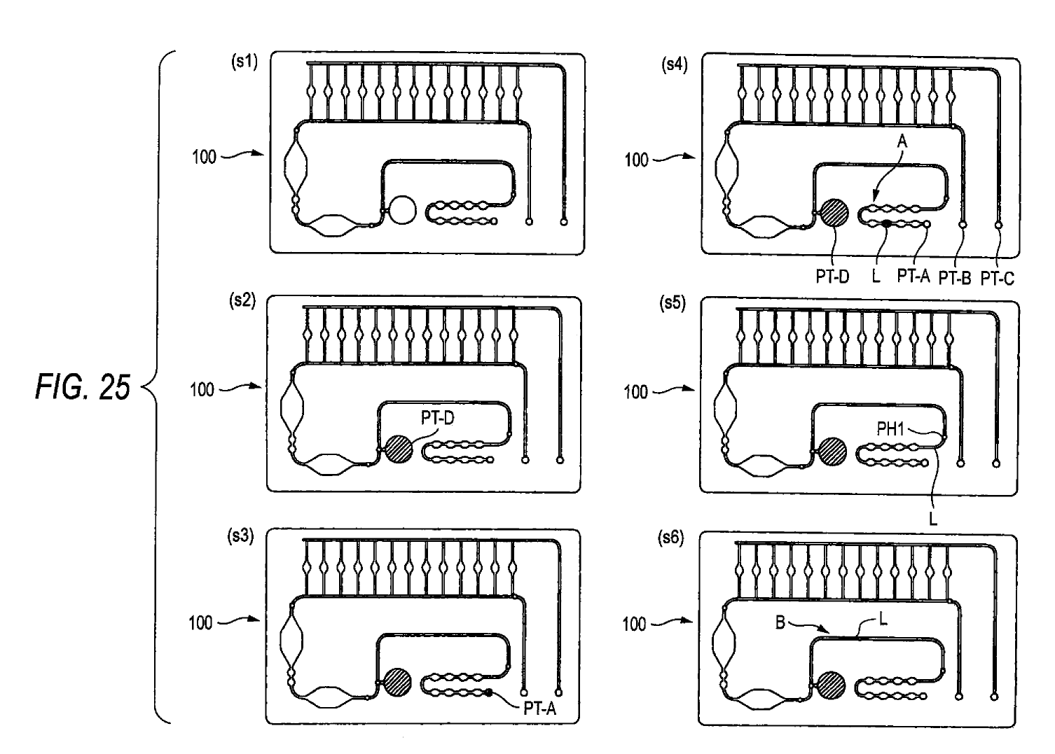

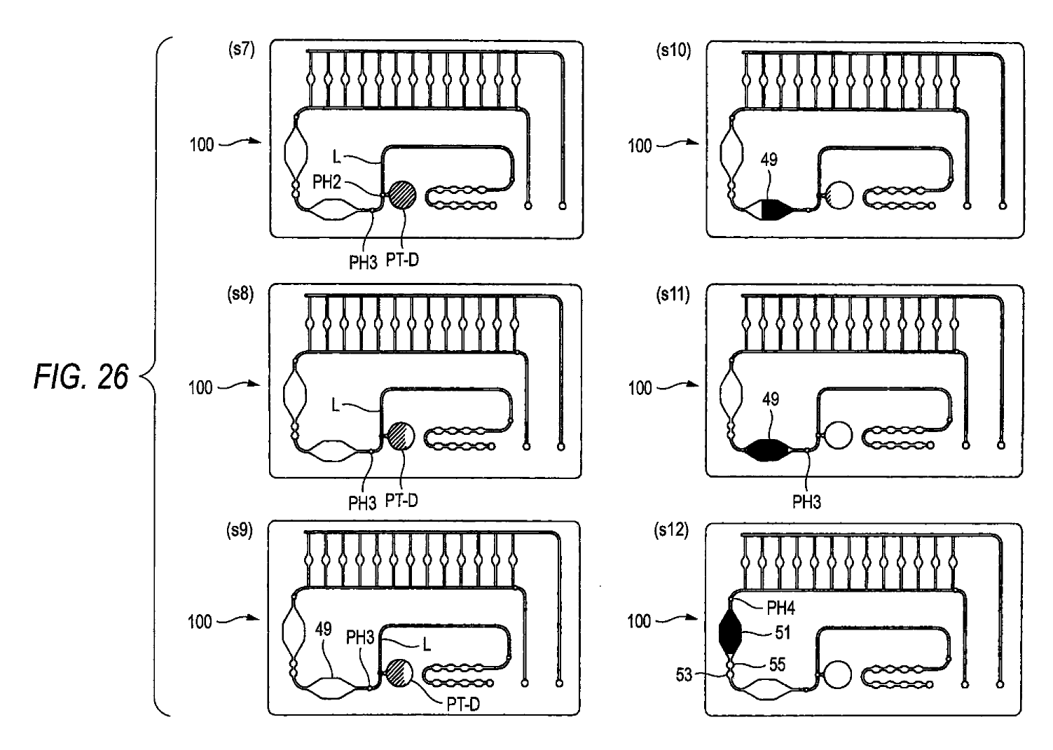

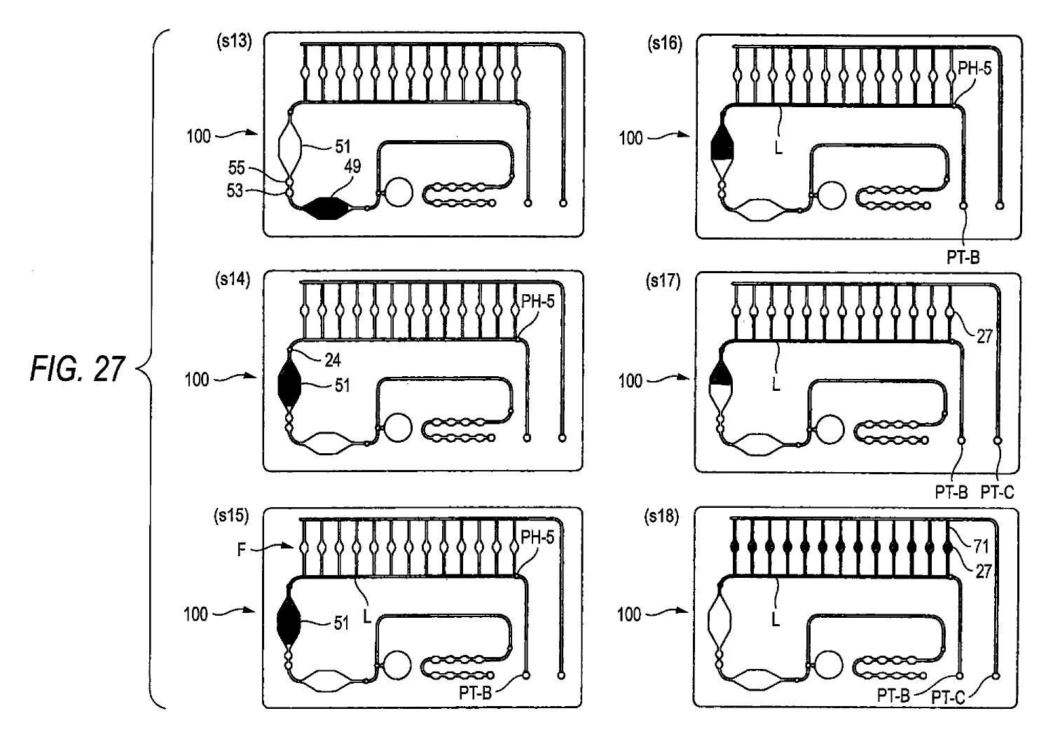

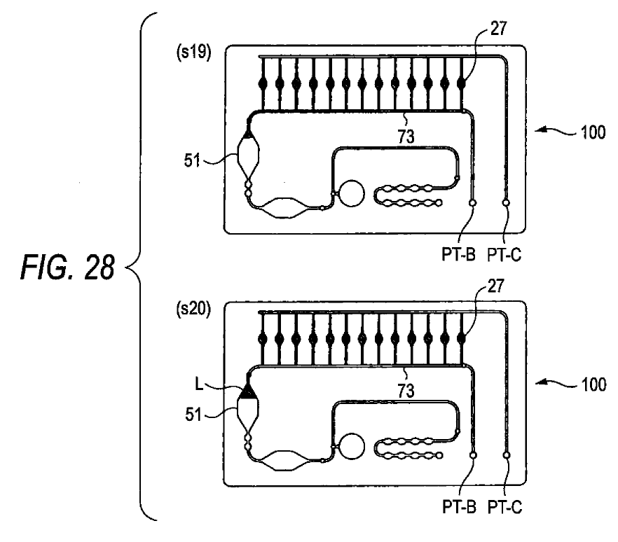

FIG. 24 is a time chart to represent the operation state of each component involved in the drive control of the microfluidic chip along the time axis, FIG. 25 is a schematic representation of the operation from liquid setting to the first heating, FIG. 26 is a schematic representation of the operation to enzyme mixing, FIG. 27 is a schematic representation of the operation to dispensing into the reaction section, and FIG. 28 is a schematic representation of the operation from dispensing to testing completion.

In the description to follow, control operation V1 to V13 in FIG. 24 and steps S1 to S20 in FIGS. 25 to 28 are associated with each other.

First, the chip 200 is prepared and a READY switch of a testing apparatus 11 is pressed (V1, S1). A reaction amplification reagent is input to a second port PT-D (S2). The magnitude relation of capillary forces of channels in the second port PT-D is port D exit channel 45>main channel 47>second port PT-D and a Laplace pressure valve is formed in the connection part of the port D exit channel 45 and the main channel 47. Thus, the reaction amplification reagent remains on the connection face of the port D exit channel 45 and the main channel 47 without flowing out to the main channel 47.

Next, blood and a pretreatment reagent are input to a first port PT-A (S3). The chip 200 is set in the testing apparatus 11 and a START switch of the testing apparatus 11 is pressed (V2). Than, port pads are pressed against the first port PT-A, the second port PT-D, a third port PT-B, and a fourth port PT-C. At this time, the pads corresponding to the first port PT-A, the second port PT-D, the third port PT-B, and the fourth port PT-C are placed in an atmospheric release state and as the pads are pressed against the ports, the liquid input to the chip does not move. Upon completion of pressing the pads against the ports, the pressure of the third port PT-B is reduced (V3) and the blood and the pretreatment reagent L pass through the sample mixing section A at high speed (100 μL/min), whereby they are fixed uniformly (S4). The second port PT-D is sucked as the same pressure reduction as the third port PT-B and it liquid delivery resistance of the blood and the pretreatment reagent is large, the pretreatment reagent in the second port PT-D does not flow out into the channel.

The liquid arrives at a sensing position PH1, a sensor PH-1 of a liquid position detection section is turned ON (V4) and then the suction speed is switched to low speed (30 μL/min) (S5).

The liquid passes through a heated section B at low speed (30 μL/min) (S6), whereby the mixed liquid L of the blood and the pretreatment reagent is heated to 98° C. for a given time (for example, 15 seconds), and DNA in leucocytes is extracted, resulting in one strand.

When the liquid arrives at a sensing position PH2 and a sensor PH-2 is turned ON (V5), the second port PT-D is placed in an atmospheric release state and at the same time, the first port PT-A is closed and the reaction amplification reagent flows out only from the second port PT-D by suction (S7) and converges into the mixed liquid L of the blood and the pretreatment reagent without containing any bubble (S8).

When the liquid arrives at a sensing position PH3 and a sensor PH-3 is turned ON (V5), the suction speed is switched to high speed (100 μL/min) and a given flow quantity (45 μL) is sucked (S9, S10).

The first port PT-A is placed in an atmospheric release state (V7), further 15 μL is sucked, the second port PT-D becomes empty, and the liquid is mixed in the first mixing section 49 (S11).

Further, 80 μL is sucked at higher speed (500 μL/min) (V8), whereby the mixed liquid L passes through the first retention section 53 and the second retention section 55 and the enzyme is dissolved and the liquid is mixed in the second mixing section 51 (S12).

Further, 80 μL is pressurized at high speed (500 μL/min) (V9), whereby the mixed liquid L is transported to the first mixing section 49 and undissolved enzyme is dissolved and the liquid is mixed in the first mixing section 49 (S13).

Further, 80 μL is sucked at high speed (500 μL/min) (V10), whereby the enzymes in the first retention section 53 and the second retention section 55 are completely dissolved and the liquid is mixed in the second mixing section 51 (S14).

Similar reciprocating operation is also performed in the following reciprocating mixing channel part, whereby the liquid is mixed uniformly.

Next, suction is executed at 0.2 kPa low speed (30 μL/min) from the third port PT-D (V11), whereby the mixed liquid L in the second mixing section 51 is transported to the channel of a reaction section F (S15).

When the liquid arrives at a sensing position PH5 and a sensor PH-5 is turned ON (V12), the third port PT-B is placed in a closed state, the fourth port PT-C is sucked at 0.3 kPa low speed (50 μL/min), and the state is kept for five seconds. The mixed liquid L is transported into the reaction detection cell 27 and stops at a narrow reaction detection cell exit channel 71 downstream from the cell (S16, S17, and S18). When the pressure sensor PS reaches a given pressure, it can be determined that dispensing is complete.

At this time, each reaction detection cell 27 is kept at the normal temperature and the primer previously immobilized with gelatin is retained in the cell without dissolving.

Next, the fourth port PT-C is placed in a closed state (V13) and is pressurized at speed of 200 μL/min from the third port PT-B, whereby the mixed liquid in a main channel 73 joining the reaction detection cells 27 is pushed back to the second mixing section 51 (S19) and the mixed liquid L is weighed 2.5 μL and is dispensed to each reaction detection cell 27 and they are placed in a state in which they are not joined by liquid (S20).

As described above, similar functions and effects to those of the first embodiment can also be provided according to the configuration of the embodiment provided by simplifying the configuration of the fifth channel (enzyme mixing section) E.

The microfluidic chip according to the invention includes channels for mixing-various reagents and dispensing a fixed quantity of the mixed liquid as component measures in addition to the first port for inputting sample liquid and a pretreatment reagent, the second port for inputting a reaction amplification reagent, and the third port for supplying air pressure to the channel, whereby it is made possible to perform complicated handling of limited liquid by pneumatic drive from the outside of the chip particularly with simple channels not containing any active valve or pump. Thus, liquid delivery control is made possible according to a simple structure and simply by inputting a sample and a liquid reagent, automatically any desired droplet operation and chemical reaction are conducted and the need for intricate operation of pipetting, taking out from, taking to the device, etc., is eliminated and the high analysis result can be obtained.

The entire disclosure of each and every foreign patent application from which the benefit of foreign priority has been claimed in the present application is incorporated herein by reference, as if fully set forth.

What is claimed is:

1. A microfluidic chip including channels for detecting a plurality of types of nucleic acid sequences, comprising:
a first port for inputting: a sample liquid containing a biological cell; and a first liquid;
a second port for inputting a second liquid;
a third port for supplying air pressure to the channels;
a first channel (A) for mixing the sample liquid and the first liquid input from the first port to generate a first mixed liquid;
a second channel (B) for heating the first mixed liquid;
a third channel (C) for allowing the second liquid to converge into the first mixed liquid treated in the second channel (B) to generate a second mixed liquid;
a fourth channel (D) installing a first solid that dissolves with the passage of the second mixed liquid converged in the third channel (C);
a fifth channel (E) for promoting mixing of the first solid into the second mixed liquid treated in the fourth channel (D);
a plurality of sixth channels (F) each having a second solid solidified and installed in the sixth channel (F); and
a seventh channel (G), which connects the fifth channel (E) and the plurality of sixth channels (F), for dispensing a fixed quantity of the second mixed liquid treated in the fifth channel (E) to each of the plurality of sixth channels (F).

2. The microfluidic chip according to claim 1,
wherein the first liquid comprises a pretreatment reagent.

3. The microfluidic chip according to claim 1,
wherein the second liquid comprises a reaction amplification reagent.

4. The microfluidic chip according to claim 1,
wherein an enzyme is mixed into the first solid

5. The microfluidic chip according to claim 1,
wherein a primer is mixed into the second solid.

6. The microfluidic chip according to claim 1, which is a microfluidic chip for detecting presence or absence of a plurality of types of nucleic acid sequences contained in a blood.

7. The microfluidic chip according to claim 1, which is a microfluidic chip for detecting presence or absence of a single nucleotide polymorphism.

8. The microfluidic chip according to claim 1,
wherein DNA amplification reaction is executed isothermally in the sixth channel (F).

9. The microfluidic chip according to claim 8, which has light-transmitting property enable to detect fluorescence occurring in the DNA amplification.

10. The microfluidic chip according to claim 1,
wherein the first channel (A) comprises an alternating pattern of: wide channel parts each with a cross-section area in an orthogonal direction to a flow direction of a liquid being larger than cross-sectional areas in any other channels in the first channel (A); and narrow channel parts each having a smaller cross-sectional area than the wide channel parts.

11. The microfluidic chip according to claim 1,
wherein the third channel (C) comprises:
a port for retaining the second liquid;
a main channel where the first mixed liquid is delivered; and
a port exit channel disposed at a midpoint in the main channel for allowing the main channel to communicate with the port, and
wherein magnitude relation of capillary forces is:
port exit channel>main channel>port.

12. The microfluidic chip according to claim 1,
wherein the fourth channel (D) comprises;
a retention section for installing the first solid, and
channels placed in upstream and downstream sides of the retention section each having a narrower width than the retention section.

13. The microfluidic chip according to claim 1,
wherein the fifth channel (E) comprises a plurality of liquid reservoir chambers, and
the fourth channel (D) is disposed between the plurality of liquid reservoir chambers, and
wherein the second mixed liquid goes and returns between the plurality of liquid reservoir chambers, so as to dissolve and mix the first solid.

14. The microfluidic chip according to claim 1,
wherein the fifth channel is provided at a midpoint in a channel from the first and second ports to the third port and comprises a first mixing section and a second mixing section placed in order from a side of the first and second ports, and
wherein each of the first mixing section and the second mixing section is formed alternately with first channel parts each with a perpendicular cross-section area in a flow direction of a liquid being larger than perpendicular cross-sectional areas in any other channels in each of the first mixing section and the second mixing section; and second channel parts each having a smaller perpendicular cross-sectional area than the first channel part,
the perpendicular cross-sectional area of the first channel part in the first mixing section is formed smaller than that of the first channel part in the second mixing section, and
a channel direction length of the first channel part in the first mixing section is formed longer than a channel direction length of the first channel part in the second mixing section.

15. The microfluidic chip according to claim 1, wherein the second solid in the sixth channel (F) is solidified and placed on an upper face of the sixth channel (F).

16. The microfluidic chip according to claim 5,
wherein the primer placed in the sixth channel (F) is mixed in a substance dissolved by heating and is solidified.

17. The microfluidic chip according to claim 5,
wherein the sixth channel (F) comprises:
a reaction detection cell for retaining the primer; and
an upstream channel and a downstream channel of the reaction detection cell, and
wherein a heated region consisting of the whole reaction detection cell and parts of channels in a reaction detection cell side of the upstream channel and the downstream channel is formed thinner than any other regions in the sixth channel (F).

18. The microfluidic chip according to claim 3, further comprising:
a block member for blocking all of the first, second, and third ports to perform amplification reaction using the reaction amplification reagent in a hermetically sealed space.

19. The microfluidic chip according to claim 1,
wherein an inner face of the sixth channel (F) is a continuous smooth face for preventing formation of a minute gap space not tilled with liquid when a liquid flows through an inside of the sixth channel (F).

20. The microfluidic chip according to claim 1,
wherein an inner face of each of the channels has wettability of at least two levels or more.
